(19) United States
(12) Patent Application Publication
(10) Pub. No.: US 2026/0335237 A1
(43) Pub. Date: Oct. 29, 2026

(54) LIPID NANOPARTICLES

(71) Applicant: Pfizer Inc., New York, NY (US)

(72) Inventors: Patrick Lim Soo, Winchester, MA (US); Chelsea Renee Thorn, Cambridge, MA (US)

(21) Appl. No.: 19/111,018

(22) PCT Filed: Sep. 13, 2023
(86) PCT No.: PCT/IB2023/059101

§ 371 Date: Mar. 12, 2025

(51) Int. Cl. A61K 9/1272; A61K 9/51; A61K 31/7105; C12N 15/88

(52) U.S. Cl. CPC A61K 9/1272; A61K 9/5123; A61K 31/7105; C12N 15/88

(57) ABSTRACT

The present disclosure relates to lipids, lipid nanoparticles and formulations thereof, and to methods involving the lipids and lipid nanoparticles to deliver one or more therapeutics and/or prophylactics to and/or produce polypeptides in mammalian cells or organs.

### TECHNICAL FIELD

The present disclosure relates to lipids, lipid nanoparticles and formulations thereof, and to methods involving the lipids and lipid nanoparticles to deliver one or more therapeutics and/or prophylactics to and/or produce polypeptides in mammalian cells or organs.

### BACKGROUND

Lipid nanoparticles (LNPs) are effective drug delivery systems for biologically active compounds, such as therapeutic and/or prophylactic nucleic acids, proteins, and peptides, which are otherwise cell impermeable. Drugs based on nucleic acids, which include large nucleic acid molecules such as, e.g., in vitro transcribed messenger RNA (mRNA) as well as smaller polynucleotides that interact with mRNA or a gene, need to be delivered to the proper cellular compartment to be effective. For example, nucleic acids such as mRNA suffer from their physico-chemical properties that render them impermeable to cells. In addition, nucleic acids such as mRNA are rapidly degraded by nucleases present in blood and other fluids or in tissues and have been shown to stimulate strong immune responses in vitro and in vivo. See, e.g., Robbins et al, Oligonucleotides 19:89-102 (2009); International Patent Application Publication Number WO2016/1 18697; and Pardi et al. J. Control. Release, 217:345-351 (2015).

Lipid nanoparticle formulations have improved nucleic acid delivery in vivo. For example, such formulations have significantly reduced siRNA doses necessary to achieve target knockdown in vivo (see Zimmermann et al., Nature 441:111-114, 2006). Further, upon intramuscular administration, LNPs have been shown to enable the uptake by host cells and delivery of mRNA inside the cytosol via endocytosis into an endosome, where translation of the gene of interest can subsequently occur (see, e.g. Liang et al., Mol. Ther., 25:2635-2647 (2017)). Typically, such lipid nanoparticle drug delivery systems are multi-component formulations comprising cationic lipids, helper (or structural) lipids, and lipids containing polyethylene glycol (PEG). The positively charged cationic lipids bind to the anionic nucleic acid, while the other components support a stable self-assembly of the lipid nanoparticles. Efforts have been directed toward improving delivery efficacy of lipid nanoparticle formulations. Many such efforts have been aimed toward developing more appropriate cationic lipids. See, e.g., Akinc et al., Nature Biotechnology 26:561-569 (2008); Love et al., Proc. Natl. Acad. Sci. USA 107:1864-1869, (2010); Baigude et al., Journal of Controlled Release 107:276-287 (2005); U.S. Patent Publication No. 2020-0155691; and Semple et al., Nature Biotechnology 28:172-176 (2010). Certain types of non-cationic lipids, sometimes referred to as “helper lipids”, or “structural lipids”, are thought to mediate structural morphologies of LNPs, including non-lamellar structures. LNPs can form into an array of mesophases, including micelles, micellar cubic, hexagonal, bicontinuous cubic, lamellar, inversed bicontinuous cubic, inversed hexagonal, inversed micellar cubic and inversed micelle mesophases (see, e.g. Eygeris et al., Nono Letters 20, 4543 (2020), and Kaasgaard et al., Phys Chem Chem Phys 2006: 8 (43): 4957-75); WO2022/087686; Han et al. Nat Commun 12, 7233 (2021); and Schoenmaker et al. Int. J. of Pharmaceutics 601:120586 (2021). The structural morphology of LNPs is considered an essential factor in how the encapsulated cargo is packaged and mediates fusion to the host cell where the cargo is ultimately released, and is the subject of ongoing research (e.g. Eygeris et al., Nano Lett. 20:6, 4543-4549 (2020); Gomez-Aguado et al., Nanomaterials 10 (2): 364 (2020)).

Glyceryl monoolein is a structural lipid that has been used in forming LNP structures with cubic morphology (see, e.g. H. Kim & C. Leal, ACS Nano 9 (10): 10214-10226 (2015)). Phytantriol is an aliphatic alcohol that is known to form cubic phases at physiological temperatures (see, e.g. Barauskas et al., Langmuir 19:9562-9565 (2003); “Final Report on the Safety Assessment of Phytantriol”, Int. J. Toxicol. 26 Supp 1:107-114 (2007).

Despites these efforts, there remains a need for LNP formulations that provide high potency following administration, that allow for the administration of lower doses of nucleic acids or other cargo, and that have improved temperature stability.

### SUMMARY

Provided herewith are lipid nanoparticles (LNPs) and pharmaceutical compositions comprising the LNPs. The LNPs and pharmaceutical compositions are particularly useful for delivering a nucleic acid to a patient (e.g., a human) or to a cell. LNP formulations useful for the delivery of nucleic acids frequently employ a polymer-lipid conjugate, such as a PEG-lipid conjugate, which serves to help control particle size during LNP manufacture and prevent unwanted aggregation in the vial and in the blood after administration. The PEG-lipid conjugates also help to prevent unwanted opsonization in the blood. These PEG-lipid conjugates can use a PEG polymer component with a MW of about 2000. These PEG-lipid conjugates can be employed in molar ratios (relative to other lipids in the composition) of about 0.5% to about 10%. In addition, lower molecular weight PEG polymers (500-1,000) could be employed in molar ratios (relative to other lipids in the composition) of about 0.5% to about 10%. Also, higher molecular weight PEG polymers (5,000-20,000) could be employed in molar ratios (relative to other lipids in the composition) of about 0.2% to about 0.5% (see e.g. WO 2020219941). In addition, LNP formulations typically employ an ionizable lipid (such as a cationic lipid), and at least two structural lipids such as cholesterol and a phospholipid, such as distearoylphosphatidylcholine (DSPC). In contrast, lipid nanoparticle formulations described herein can contain at least one structural lipid selected from a monoacylglycerol and an aliphatic alcohol. For example, LNP formulations described herein can contain an ionizable lipid (such as a cationic lipid), a polymer lipid conjugate (such as a PEG-lipid conjugate), and one or two structural lipids (such as cholesterol, glyceryl monoolein, or phytantriol). Accordingly, and as described more fully herein, new formulations have been developed that use various monacylglycerol and alpiphatic alcohol structural lipids such as glyceryl monoolein and phytantriol, to provide unique structural and beneficial properties for the LNPs. For example, as described more fully herein, new LNP formulations have been developed that use either monoolein or phytantriol as the sole structural lipid, or in combination with cholesterol as the other structural lipid.

The present disclosure provides lipid nanoparticles that comprise a nucleic acid, a cationic lipid, a polymer-lipid conjugate, such as a polyethylene glycol (PEG)-lipid conjugate, and at least one structural lipid selected from a monoacylglycerol and an aliphatic alcohol, such as glyceryl monoolein and phytantriol. In addition, the LNPs can comprise additional structural lipids such as sterols, phospholipids, and other structural lipids known in the art. The present disclosure further provides methods of using such LNPs to induce an immune response against a pathogen in a subject, such as a mammal (e.g. in a human); and/or to treat or prevent a disease, disorder, or condition in a subject, such as a mammal (e.g. in a human). For example, one aspect of the present disclosure relates to the administration of a therapeutically effective amount of any of the LNPs described herein to a subject in need thereof for the treatment and/or prevention of a disease or disorder associated with coronaviruses, influenza, varicella zoster virus (shingles), respiratory syncytial virus, and other bacterial, fungal, or viral infections. In a further aspect, the present disclosure relates to methods of inducing an effective immune response against a pathogen such as those noted herein in order to effectively prevent a disease or disorder in a subject caused by said pathogen, comprising administering an effective amount to said subject of any of the LNPs of the present disclosure.

The present disclosure further provides LNPs that have a delayed release profile of the RNA contained therein as compared to LNPs that do not have a delayed release profile.

### BRIEF DESCRIPTION OF THE FIGURES

FIG. 1. The critical quality attributes (CQAs) of six LNPs over a time course period of three months at refrigerator (5 °C) and room temperature (25 °C), where A depict the encapsulation efficiency, B depict particle size, C show poly-dispersity index (PDI), and D show the RNA integrity.

FIG. 2. Representative cryo-electron micrographs of four LNPs, including A. standard DSPC:cholesterol (10:28.5 mol %), B. GMO 70 mol %, C. GMO:cholesterol 14.4:55.6 mol %, D. cholesterol:GMO 22:77 mol %, E. PHY 70 mol % and F PHY:GMO 14.4:55.6 mol %. Arrow bar for all images is the same and shown in panel B, E and F, white boxes indicate flat surfaces of LNPs, and black boxes indicate higher internal structure of LNPs.

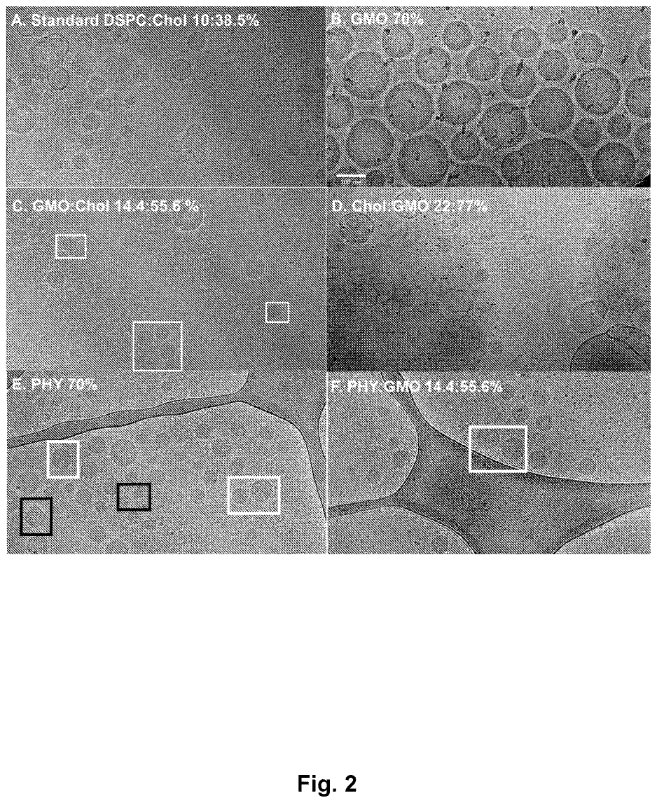

FIG. 3. SAXS diffractograms of various mRNA-LNPs FIG. 3A: LNPs containing GMO as the structural lipid (with different variations) compared to DSPC:Chol and FIG. 3B: LNPs containing PHY as structural lipid (with different variations) compared to DSPC:Chol.

FIG. 4. 1D-NMR spectrum of various mRNA-LNPs.

FIG. 5. Cyro-electron microscopy (EM) micrographs of: FIG. 5A GMO:Chol 14.4:55.6% mol and FIG. 5B. Chol:GMO 22:77% mol.

FIG. 6. Absolute HA copy numbers determined by RT-qPCR at increasing amounts of mRNA-LNPs at A. 1 h post transfection and B. 4 h post transfection for the seven different formulations.

FIG. 7. Cellular internalization determined by cytoplasmic distribution at 4 h post transfection with smFISH quantification of Cali/09/HA positive cells (A) and clusters (B). At 24 h post transfection the cytoplasmic distribution is further shown with the smFISH quantification of Cali/09/HA positive cells (C) and clusters (D).

FIG. 8. Transfection efficiency is demonstrated in: FIG. 8A—the percent positive cells expressing the HA antigen at various mRNA-LNP concentrations and formulations; FIG. 8B—the corresponding Mean fluorescence intensity (MFI) data from the various mRNA-LNP concentrations and formulations that are depicted as percent positive in FIG. 8A; and FIG. 8C—the number of cells present across the experiment after transfection with the various mRNA-LNPs concentration and formulation.

### DETAILED DESCRIPTION

Described below are a number of embodiments (E) of the present disclosure.
E1. According to a first aspect of the present disclosure, there is provided a lipid nanoparticle (LNP) comprising: (a) at least one nucleic acid; (b) at least one polymer-lipid conjugate in an amount from about 0.05 to about 5 mol % of the total lipid in the particle; (c) at least one cationic lipid in an amount from about 0.1 to about 50 mol % of the total lipid in the particle; and (d) at least one structural lipid selected from a monoacylglycerol and an aliphatic alcohol, wherein each of said at least one structural lipids is present in an amount from about 10 to about 99 mol % of the total lipid in the particle.
E2. The LNP of embodiment E1, wherein the at least one polymer-lipid conjugate is a polyethylene glycol (PEG)-lipid conjugate.
E3. The LNP of embodiment E2, wherein the PEG-lipid conjugate is present in an amount from about 0.1 to about 10 mol % of the total lipid in the particle.
E4. The LNP of embodiment E2, wherein the PEG-lipid conjugate is present in an amount from about 0.5 to about 5 mol % of the total lipid in the particle.
E5. The LNP of embodiment E2, wherein the PEG-lipid conjugate is present in an amount from about 1 to about 2 mol % of the total lipid in the particle.
E6. The LNP of embodiment E2, wherein the PEG-lipid conjugate is present in an amount of about 1.5 mol % of the total lipid in the particle.
E7. The LNP of embodiment E2, wherein the PEG-lipid conjugate is selected from: dimyristoylphosphatidylcholine-polyethylene glycol-2000 (DMPC-PEG); 1,2-dimyristoyl-rac-glycero-3-methoxypolyethylene glycol-2000 (DMG-PEG); 1,2-dioleoyl-sn-glycero-3-phosphoethanolamine-N-[amino(polyethylene glycol)-2000] ammonium salt (DOPE-PEG); 1,2-distearoyl-sn-glycero-3-phosphoethanolamine-poly(ethylene glycol)-2000 (DSPE-PEG); mPEG-N,N-ditetradecylacetamide (ALC-0159); and oleoyl polyethylene glycol 2000.
E8. The LNP of embodiment E1, wherein the at least one cationic lipid is present in an amount from about 0.5 to about 40 mol % of the total lipid in the particle.
E9. The LNP of embodiment E1, wherein the at least one cationic lipid is present in an amount from about 1 to about 35 mol % of the total lipid in the particle.
E10. The LNP of embodiment E1, wherein the at least one cationic lipid is present in an amount from about 5 to about 30 mol % of the total lipid in the particle.
E11. The LNP of embodiment E1, wherein the at least one cationic lipid is present in an amount from about 10 to about 20 mol % of the total lipid in the particle.
E12. The LNP of embodiment E1, wherein the at least one cationic lipid is selected from: (6Z,9Z,28Z,31Z)-heptatriaconta-6,9,28,31-tetraen-19-yl 4-(dimethylamino)butanoate (Dlin-MC3-DMA); [(4-hydroxybutyl)azanediyl]di(hexane-6,1-diyl)bis(2-hexyldecanoate) (ALC-0315); 2-[2,2-bis[(9Z,12Z)-octadeca-9,12-dienyl]-1,3-dioxolan-4-yl]-N,N-dimethylethanamine (DLin-KC2-DMA); 1,2-dilinoleyloxy-n,n-dimethyl-3-aminopropane (DLinDMA); N,N-dimethyl-2,3-bis[(Z)-octadec-9-enoxy]propan-1-amine (DODMA); 5-carboxyspermylglycinedioctadecylamide (DOGS); and 1,2-dioleoyl-3-dimethylammonium-propane (DODAP).
E13. The LNP of embodiment E12, wherein the at least one cationic lipid is Dlin-MC3-DMA.
E14. The LNP of embodiment E1, wherein the at least one structural lipid is glyceryl monoolein (GMO).
E15. The LNP of embodiment E14, wherein GMO is present in an amount from about 10 to about 99 mol % of the total lipid in the particle.
E16. The LNP of embodiment E14, wherein GMO is present in an amount from about 20 to about 90 mol % of the total lipid in the particle.
E17. The LNP of embodiment E14, wherein GMO is present in an amount from about 30 to about 80 mol % of the total lipid in the particle.
E18. The LNP of embodiment E14, wherein GMO is present in an amount from about 40 to about 70 mol % of the total lipid in the particle.
E19. The LNP of embodiment E14, wherein GMO is present in an amount from about 50 to about 60 mol % of the total lipid in the particle.
E20. The LNP of embodiment E14, wherein GMO is present in an amount from about 10, about 20, about 30, about 40, about 50, about 60, about 70, about 80, about 90 or about 99 mol % of the total lipid in the particle.
E21. The LNP of embodiment E1, wherein the at least one structural is phytantriol (PHY).
E22. The LNP of embodiment E21, wherein PHY is present in an amount from about 10 to about 99 mol % of the total lipid in the particle.
E23. The LNP of embodiment E21, wherein PHY is present in an amount from about 20 to about 90 mol % of the total lipid in the particle.
E24. The LNP of embodiment E21, wherein PHY is present in an amount from about 30 to about 80 mol % of the total lipid in the particle.
E25. The LNP of embodiment E21, wherein PHY is present in an amount from about 40 to about 70 mol % of the total lipid in the particle.
E26. The LNP of embodiment E21, wherein PHY is present in an amount from about 50 to about 60 mol % of the total lipid in the particle.
E27. The LNP of embodiment E21, wherein PHY is present in an amount from about 10, about 20, about 30, about 40, about 50, about 60, about 70, about 80, about 90 or about 99 mol % of the total lipid in the particle.
E28. The LNP of embodiment E1, comprising at least one monoacylglycerol structural lipid and at least one aliphatic alcohol structural lipid.
E29. The LNP of embodiment E28, wherein the monoacylglycerol is GMO.
E30. The LNP of embodiment E28, wherein the aliphatic alcohol is PHY.
E31. The LNP of embodiment E28, wherein the monoacylglycerol is GMO, and the aliphatic alcohol is PHY.
E32. The LNP of embodiment E31, wherein GMO and PHY are each present in an amount from about 10 to about 98.5 mol % of the total lipid in the particle.
E33. The LNP of embodiment E1, further comprising at least one additional structural lipid that is not a monoacylglycerol or an aliphatic alcohol, wherein said at least one additional structural lipid is present in an amount from about 10 to about 75 mol % of the total lipid present in the particle.
E34. The LNP of embodiment E33, wherein said at least one additional structural lipid is a sterol.
E35. The LNP of embodiment E34, wherein said sterol is cholesterol.
E36. The LNP of embodiment E33, wherein said at least one additional structural lipid is a phospholipid.
E37. The LNP of embodiment E36, wherein said phospholipid is distearoylphosphatidylcholine (DSPC).
E38. The LNP of embodiment E35, wherein cholesterol is present in an amount from about 10 to about 75 mol % of the total lipid present in the particle.
E39. The LNP of embodiment E37, wherein DSPC is present in an amount from about 10 to about 75 mol % of the total lipid present in the particle.
E40. The LNP of embodiment E33, wherein: the at least one cationic lipid is present in an amount of about 28.5 mol %; the at least one structural lipid is GMO and is present in an amount of about 14.4 mol %; the at least one additional structural lipid that is not a monoacylglycerol or an aliphatic alcohol is cholesterol and is present in an amount of about 55.6 mol %; and the at least one polymer-lipid conjugate is DOPE-PEG and is present in an amount of about 1.5 mol %.
E41. The LNP of embodiment E33, wherein: the at least one cationic lipid is present in an amount of about 1.5 mol %; the at least one structural lipid is GMO and is present in an amount of about 77 mol %; the at least one additional structural lipid that is not a monoacylglycerol or an aliphatic alcohol is cholesterol and is present in an amount of about 20 mol %; and the at least one polymer-lipid conjugate is DOPE-PEG and is present in an amount of about 1.5 mol %.
E42. The LNP of embodiment E1, wherein: the at least one cationic lipid is present in an amount of about 28.5 mol %; the at least one structural lipid is PHY and is present in an amount of about 70 mol %; and the at least one polymer-lipid conjugate is DMG-PEG and is present in an amount of about 1.5 mol %.
E43. The LNP of embodiment E1, wherein: the at least one cationic lipid is present in an amount of about 28.5 mol %; the at least one structural lipid is GMO and is present in an amount of about 14.4 mol %; a second structural lipid is PHY and is present in an amount of about 55.6 mol %; and the at least one polymer-lipid conjugate is DOPE-PEG and is present in an amount of about 1.5 mol %.
E44. The LNP of embodiment E1, wherein: the at least one cationic lipid is present in an amount of about 28.5 mol %; the at least one structural lipid is PHY and is present in an amount of about 14.4 mol %; a second structural lipid is GMO and is present in an amount of about 55.6 mol %; and the at least one polymer-lipid conjugate is DMG-PEG and is present in an amount of about 1.5 mol %.
E45. The LNP of embodiment E1, wherein: the at least one cationic lipid is present in an amount of about 28.5 mol %; the at least one structural lipid is GMO and is present in an amount of about 70 mol %; and the at least one polymer-lipid conjugate is DOPE-PEG and is present in an amount of about 1.5 mol %.
E46. The LNP of any one of embodiments E1-E45, wherein the particle size of the LNP is less than about 500 nm.
E47. The LNP of embodiment E46, wherein the particle size of the LNP is from about 20 to about 500 nm.
E48. The LNP of embodiment E47, wherein the particle size of the LNP is from about 20 to about 300 nm.
E49. The LNP of embodiment E48, wherein the particle size of the LNP is from about 20 to about 200 nm.
E50. The LNP of embodiment E49, wherein the particle size of the LNP is from about 20 to about 150 nm.
E51. The LNP of embodiment E50, wherein the particle size of the LNP is from about 20 to about 100 nm.
E52. The LNP of embodiment E49, wherein the particle size of the LNP is about 20, about 30, about 40, about 50, about 60, about 70, about 80, about 90, about 100, about 110, about 120, about 130, about 140, about 150, about 160, about 170, about 180, about 190, or about 200 nm.
E53. A composition comprising the LNPs of any one of embodiments E1-E45, wherein the average particle size of the LNPs is less than about 500 nm.
E54. The composition of embodiment E53, wherein the average particle size of the LNPs is from about 20 to about 500 nm.
E55. The composition of embodiment E54, wherein the average particle size of the LNPs is from about 20 to about 300 nm.
E56. The composition of embodiment E55, wherein the average particle size of the LNPs is from about 20 to about 200 nm.
E57. The composition of embodiment E56, wherein the average particle size of the LNPs is from about 20 to about 150 nm.
E58. The composition of embodiment E57, wherein the average particle size of the LNPs is from about 20 to about 100 nm.
E59. The composition of embodiment E56, wherein the average particle size of the LNPs is about 20, about 30, about 40, about 50, about 60, about 70, about 80, about 90, about 100, about 110, about 120, about 130, about 140, about 150, about 160, about 170, about 180, about 190, or about 200 nm.
E60. The LNP of any one of embodiments E1-E45, wherein the nucleic acid is DNA, plasmid DNA, minicircle DNA, ceDNA (closed ended DNA), SIRNA, mRNA, miRNA, self-replicating RNA, CRISPR RNA, a gene editing construct, an RNA editing construct, a base editing construct, or a prime editing construct.
E61. The LNP of any one of embodiments E1-E45, wherein the at least one nucleic acid is mRNA.
E62. A pharmaceutical composition comprising the LNP of any one of embodiments of E1-E45, and a pharmaceutically acceptable carrier.
E63. The pharmaceutical composition of embodiment E62, which is formulated for intravenous, intramuscular, subcutaneous, intratumoral, intranasal, or inhalation administration.
E64. A method for delivering a nucleic acid to a cell comprising contacting the cell with the LNP of any one of embodiments E1-E45, or E60-E61.
E65. A method for delivering a nucleic acid of interest to a subject comprising administering to the subject at least one dose of the composition of embodiment E62.
E66. The method of embodiment E65, wherein the nucleic acid is mRNA that encodes at least one protein of interest.
E67. The method of embodiment E66, wherein the at least one protein of interest is a therapeutic protein.
E68. The method of embodiment E66, wherein the at least one protein of interest is a vaccine antigen.
E69. A method for treating a disease characterized by a genetic defect that results in a deficiency of a functional protein, the method comprising: administering to a subject having the disease, the LNP of embodiment E66, wherein the mRNA encodes the functional protein or a protein having the same biological activity as the functional protein.
E70. A method for inducing an immune response against a pathogen in a subject comprising administering to the subject the LNP of embodiment E66, wherein the mRNA encodes a protein antigen from said pathogen, or an immunogenic variant or fragment thereof.
E71. The LNP of any one of embodiments E1-E45, for the therapeutic or prophylactic treatment of a disease characterized by a genetic defect that results in a deficiency of a functional protein.
E72. The LNP of any one of embodiments E1-E45, for the therapeutic or prophylactic treatment of a disease characterized by overexpression of a polypeptide.
E73. The LNP of any one of embodiments E1-E45, for the prophylactic treatment of a disease caused by a pathogen.

### DEFINITIONS

Throughout this application, the term “about” is used according to its plain and ordinary meaning in the area of lipid chemistry and formulations to indicate a deviation of ±10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The use of the word “a” or “an” when used in conjunction with the term “comprising” may mean “one,” but it is also consistent with the meaning of “one or more,” “at least one,” and “one or more than one.”

The phrase “and/or” means “and” or “or.” To illustrate, A, B, and/or C includes: A alone, B alone, C alone, a combination of A and B, a combination of A and C, a combination of B and C, or a combination of A, B, and C. In other words, “and/or” operates as an inclusive or.

The terms “administer”, “administration”, or “administer”, and the like, describe the introduction of the relevant LNP or composition to a subject, such as a mammal (e.g. a human) by a particular route or vehicle. Routes of administration may include, but are not limited to, topical, parenteral and enteral which include oral, buccal, sub-lingual, nasal, anal, gastrointestinal, subcutaneous, intramuscular and intradermal routes of administration.

The terms “treat”, “treatment”, or “treating”, and the like, describe the administration of the relevant LNP or composition to a subject, such as a mammal (e.g. a human) to at least ameliorate, reduce or suppress existing signs or symptoms of a disease, disorder or condition experienced by the subject, to the extent that the medical condition is improved according to clinically acceptable standard(s). For example, “to treat a bacterial infection” means to reduce the infection, or eradicate the infection, or relieve symptoms of the infection in a subject, wherein the improvement and relief are evaluated with a clinically acceptable standardized test and/or an empirical test, including swab sample testing and the like.

As used herein, the terms “prevent”, “preventing” or “preventative” mean prophylactically administering the relevant LNP or composition to a subject, such as a mammal (e.g. a human) who does not exhibit signs or symptoms of a disease disorder or condition, but who is expected or anticipated to likely exhibit such signs or symptoms in the absence of prevention. Preventative treatment may at least lessen or partly ameliorate expected symptoms or signs.

As used herein, “effective amount” or “therapeutically effective amount” refers to the administration of an amount of the relevant particle or composition sufficient to prevent the occurrence of symptoms of the condition being treated, or to bring about a halt in the worsening of symptoms or to treat and alleviate or at least reduce the severity of the symptoms. The effective amount will vary in a manner which would be understood by a person of skill in the art with patient age, sex, weight etc. An appropriate dosage or dosage regime can be ascertained through routine trial or based on current treatment regimes for the active being delivered via an LNP of the present disclosure.

As used herein, the terms “subject” or “individual” or “patient” may refer to any subject, particularly a vertebrate subject, and even more particularly a mammalian subject, for whom therapy is desired. Suitable vertebrate animals include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment for a disease, disorder or condition as described herein. However, it will be understood that the aforementioned terms do not imply that symptoms are necessarily present.

A “nucleic acid,” as used herein, is a molecule comprising nucleic acid components and refers to DNA or RNA molecules. It may be used interchangeably with the term “polynucleotide”. A nucleic acid molecule is a polymer comprising or consisting of nucleotide monomers, which are covalently linked to each other by phosphodiester-bonds of a sugar/phosphate-backbone. Nucleic acids may also encompass modified nucleic acid molecules, such as, for example, base-modified, sugar-modified or backbone-modified DNA or RNA molecules. Nucleic acids can exist in a variety of forms such as: isolated segments and recombinant vectors of incorporated sequences or recombinant polynucleotides encoding polypeptides, such as antigens or one or both chains of an antibody, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, mRNA, saRNA, and complementary sequences of the foregoing described herein.

Nucleic acids can be single-stranded or double-stranded and can comprise RNA and/or DNA nucleotides and artificial variants thereof (e.g., peptide nucleic acids). In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. A tag or other heterologous polypeptide may be added to the modified polypeptide-encoding sequence, wherein “heterologous” refers to a polypeptide that is not the same as the modified polypeptide.

The term “polynucleotide” refers to a nucleic acid molecule that can be recombinant or has been isolated from total genomic nucleic acid. Included within the term “polynucleotide” are oligonucleotides (nucleic acids that are 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA, or synthetic), analogs thereof, or a combination thereof. Additional coding or non-coding sequences may, but need not, be present within a polynucleotide.

In certain aspects, there are polynucleotide variants having substantial identity to the sequences disclosed herein; those comprising equal to any one of, at least any one of, at most any one of, or between any two of 70%, 75%, 80%, 85%, 90%, 95%, 96%, 97%, 98%, or 99% or higher sequence identity, compared to a polynucleotide sequence provided herein using the methods described herein (e.g., BLAST analysis using standard parameters). In certain aspects, the isolated polynucleotide will comprise a nucleotide sequence encoding a polypeptide that has at least 90% identity to an amino acid sequence described herein, over the entire length of the sequence; or a nucleotide sequence complementary to said isolated polynucleotide. In some aspects, the isolated polynucleotide will comprise a nucleotide sequence encoding a polypeptide that has at least 95% identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, equal to any one of, at least any one of, at most any one of, or between any two of 5, 10, 15, 20, 25, 30, 35, 40, 45, 50, 75, 100, 125, 175, 200, 250, 300, 350, 400, 450, 500, 750, 1000, 1500, 3000, 5000, 6000, 7000, 8000, 9000, 10000, 11000, 12000, 13000, 14000, 15000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length being limited by the ease of preparation and use in the intended recombinant nucleic acid protocol.

In this respect, the term “gene” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar polypeptide.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, etc.); (3) translation of an RNA into a polypeptide or protein; and/or (4) post-translational modification of a polypeptide or protein.

In general, the term “engineered” refers to the aspect of having been manipulated by the hand of man. For example, a polynucleotide is considered to be “engineered” when two or more sequences that are not linked together in that order in nature are manipulated by the hand of man to be directly linked to one another in the engineered polynucleotide and/or when a particular residue in a polynucleotide is non-naturally occurring and/or is caused through action of the hand of man to be linked with an entity or moiety with which it is not linked in nature.

The term “DNA,” as used herein, means a nucleic acid molecule comprising as deoxy-adenosine-monophosphate, deoxy-thymidine-nucleotides such monophosphate, deoxy-guanosine-monophosphate and deoxy-cytidine-monophosphate monomers which are composed of a sugar moiety (deoxyribose), a base moiety and a phosphate moiety, and polymerize by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA sequence. DNA may be single stranded or double stranded. In the double stranded form, the nucleotides of the first strand typically hybridize with the nucleotides of the second strand, e.g. by A/T-base-pairing and G/C-base-pairing. DNA can contain all, or a majority of, deoxyribonucleotide residues. As used herein, the term “deoxyribonucleotide” means a nucleotide lacking a hydroxyl group at the 2′ position of a β-D-ribofuranosyl group. Without any limitation, DNA can encompass double stranded DNA, antisense DNA, single stranded DNA, isolated DNA, synthetic DNA, DNA that is recombinantly produced, and modified DNA.

The term “RNA,” as used herein, means a nucleic acid molecule comprising nucleotides such as adenosine-monophosphate, uridine-monophosphate, guanosine-monophosphate and cytidine-monophosphate monomers which are connected to each other along a so-called backbone. The backbone is formed by phosphodiester bonds between the sugar, i.e. ribose, of a first and a phosphate moiety of a second, adjacent monomer. RNA may be obtainable by transcription of a DNA-sequence, e.g., inside a cell. In eukaryotic cells, transcription is typically performed inside the nucleus or the mitochondria. In vivo, transcription of DNA may result in premature RNA which is processed into messenger-RNA (mRNA). Processing of the premature RNA, e.g. in eukaryotic organisms, comprises various posttranscriptional modifications such as splicing, 5′ capping, polyadenylation, export from the nucleus or the mitochondria. Mature messenger RNA is processed and provides the nucleotide sequence that may be translated into an amino acid sequence of a peptide or protein. A mature mRNA may comprise a 5′ cap, a 5′ UTR, an open reading frame, a 3′ UTR and a poly-A tail sequence. RNA can contain all, or a majority of, ribonucleotide residues. As used herein, the term “ribonucleotide” means a nucleotide with a hydroxyl group at the 2′ position of a β-D-ribofuranosyl group. In one aspect, RNA can be messenger RNA (mRNA) that relates to a RNA transcript which encodes a peptide or protein. As known to those of skill in the art, mRNA generally contains a 5′ untranslated region (5′ UTR), a polypeptide coding region, and a 3′ untranslated region (3′ UTR). Without any limitation, RNA can encompass double stranded RNA, antisense RNA, single stranded RNA, isolated RNA, synthetic RNA, RNA that is recombinantly produced, and modified RNA (modRNA).

An “isolated RNA” is defined as an RNA molecule that can be recombinant or has been isolated from total genomic nucleic acid. An isolated RNA molecule or protein can exist in substantially purified form, or can exist in a non-native environment such as, for example, a host cell.

A “modified RNA” or “modRNA” refers to an RNA molecule having at least one addition, deletion, substitution, and/or alteration of one or more nucleotides as compared to naturally occurring RNA. Such alterations can refer to the addition of non-nucleotide material to internal RNA nucleotides, or to the 5′ and/or 3′ end(s) of RNA. In one aspect, such modRNA contains at least one modified nucleotide, such as an alteration to the base of the nucleotide. For example, a modified nucleotide can replace one or more uridine and/or cytidine nucleotides. For example, these replacements can occur for every instance of uridine and/or cytidine in the RNA sequence, or can occur for only select uridine and/or cytidine nucleotides. Such alterations to the standard nucleotides in RNA can include non-standard nucleotides, such as chemically synthesized nucleotides or deoxynucleotides. For example, at least one uridine nucleotide can be replaced with 1-methylpseudouridine in an RNA sequence. Other such altered nucleotides are known to those of skill in the art. Such altered RNAs are considered analogs of naturally-occurring RNA. In some aspects, the RNA is produced by in vitro transcription using a DNA template, where DNA refers to a nucleic acid that contains deoxyribonucleotides. In some aspects, the RNA can be replicon RNA (replicon), in particular self-replicating RNA, or self-amplifying RNA (saRNA).

As contemplated herein, without any limitations, RNA can be used as a therapeutic modality to treat and/or prevent a number of conditions in mammals, including humans. Methods described herein comprise administration of the RNA described herein to a mammal, such as a human. For example, in one aspect such methods of use for RNA include an antigen-coding RNA vaccine to induce robust neutralizing antibodies and accompanying/concomitant T-cell response to achieve protective immunization. In some aspects, minimal vaccine doses are administered to induce robust neutralizing antibodies and accompanying/concomitant T-cell response to achieve protective immunization. In one aspect, the RNA administered is in vitro transcribed RNA. For example, such RNA can be used to encode at least one antigen intended to generate an immune response in said mammal. Pathogenic antigens are peptide or protein antigens derived from a pathogen associated with infectious disease.

The term “antigen” may refer to a substance, which is capable of being recognized by the immune system, e.g., by the adaptive immune system, and which is capable of eliciting an antigen-specific immune response, e.g. by formation of antibodies and/or antigen-specific T cells as part of an adaptive immune response. An antigen may be or may comprise a peptide or protein, which may be presented by the MHC to T-cells. An antigen may be the product of translation of a provided nucleic acid molecule, e.g., an RNA molecule comprising at least one coding sequence as described herein. In addition, fragments, variants and derivatives of an antigen, such as a peptide or a protein, comprising at least one epitope are understood as antigens.

As used herein, the term “lipid” refers to a chemical compound that is insoluble in water and extractable with an organic solvent. Lipids may be naturally occurring, or synthetic. Typically, a lipid is a biological compound. Compounds other than those specifically described herein are understood by one of skill in the art as lipids, and are encompassed by the compositions and methods of the present disclosure. A lipid component and a non-lipid may be attached to one another, either covalently or non-covalently.

As used herein, the term “lipid nanoparticle”, or “LNP”, refers to particles of any morphology generated when a cationic lipid and optionally one or more further lipids are combined, e.g. in an aqueous environment and/or in the presence of a nucleic acid such as RNA. In some aspects, lipid nanoparticles are included in a formulation that can be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid. Such lipid nanoparticles typically comprise a cationic lipid and one or more excipients, e.g., one or more neutral lipids, charged lipids, steroids, polymer conjugated lipids, or combinations thereof. In some aspects, the active agent or therapeutic agent, such as a nucleic acid (e.g., mRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated. The LNPs disclosed herein typically have a mean diameter of from about 1 nm to about 500 nm. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

The term “polymer-lipid conjugate” refers to a molecule comprising both a lipid portion and a polymer portion. An example of a polymer-lipid conjugate is a pegylated lipid. The term “pegylated lipid” (PEG lipid) refers to a molecule comprising both a lipid portion and a polyethylene glycol portion. Pegylated lipids are known in the art and include lipids such as 1-(monomethoxy-polyethyleneglycol)-2,3-dimyristoylglycerol (PEG-s-DMG), 2-[(polyethylene glycol)-2000]-N,N-ditetradecylacetamide, polyethylene glycol conjugated lipids in C14-C18 range, i.e. DMPC-PEG (Dimyristoylphosphatidylcholine-polyethylene glycol-2000), DMG-PEG (1,2-dimyristoyl-rac-glycero-3-methoxypolyethylene glycol-2000), DOPE-PEG (1,2-dioleoyl-sn-glycero-3-phosphoethanolamine-N-[amino(polyethylene glycol)-2000] ammonium salt), DSPE-PEG (1,2-Distearoyl-sn-glycero-3-phosphoethanolamine-Poly (ethylene glycol)-2000), ALC-0159, oleoyl polyethylene glycol 2000, and poly(ethylene glycol)-block-poly(propylene glycol)-block-poly(ethylene glycol), and the like. Other examples of polymer-lipid conjugates include polyoxazolines, poly(2-methyl-2-oxazoline) (PMOZ), poly(2-ethyl-2-oxazoline) (PEOZ), polysarcosines, polyglycerol polymer conjugates (i.e. PG-30), and polycarbonate polymer conjugates (i.e. PCs) and the like.

As used herein, a “cationic lipid” refers to a lipid or lipid like material having a net positive charge. Cationic lipids bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA. In some aspects, cationic lipids are ionizable such that they can exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids are comprised by the term “cationic lipid” unless contradicted by the circumstances. Without limitation, examples of cationic lipids include Dlin-MC3-DMA ((6Z,9Z,28Z,31Z)-Heptatriaconta-6,9,28,31-tetraen-19-yl 4-(dimethylamino)butanoate), ALC-0315 ([(4-hydroxybutyl)azanediyl]di(hexane-6,1-diyl)bis(2-hexyldecanoate)), DLin-KC2-DMA (2-[2,2-bis[(9Z,12Z)-octadeca-9,12-dienyl]-1,3-dioxolan-4-yl]-N,N-dimethylethanamine), DLinDMA (1,2-dilinoleyloxy-n,n-dimethyl-3-aminopropane), DODMA (N,N-dimethyl-2,3-bis[(Z)-octadec-9-enoxy]propan-1-amine), DOGS (5-carboxyspermylglycinedioctadecylamide), DODAP (1,2-Dioleoyl-3-Dimethylammonium-Propane), DOSPA (2,3-dioleyloxy-N-[2 (spermine-carboxamido)ethyl]-N,N-dimethyl-1-propanaminium), DOTAP (1,2-Dioleoyl-3-Trimethylammonium-Propane), DSDMA (1,2-distearyloxy-N,N-dimethyl-3-aminopropane), DLenDMA (1,2-dilinolenyloxy-N,N-dimethyl-3-aminopropane), DODAC (N-dioleyl-N,N-dimethylammonium chloride), DDAB (N,N-distearyl-N,N-dimethylammonium bromide), DMRIE (N-[1,2-dimyristyloxyprop-3-yl]-N,N-dimethyl-N-hydroxyethyl ammonium bromide), CLinDMA (3-dimethylamino-2-(cholest-5-en-3-beta-oxybutan-4-oxy)-1-(cis,cis-9,12-oc-tadecadienoxy)propane), CpLinDMA (2-[5′-(cholest-5-en-3-beta-oxy)-3′-oxapentoxy)-3-dimethy 1-1-(cis,cis-9′,1-2′-octadecadienoxy)propane), and DMOBA (N,N-dimethyl-3,4-dioleyloxybenzylamine).

As used herein, a “structural lipid” is a lipid that helps stabilize the formation of LNPs during their formation, and can mediate the morphology of an LNP. Such structural lipids (sometimes referred to as “helper lipids”) include non-cationic lipids, e.g., neutral lipids and anionic lipids (e.g. phospholipids). Without limitation, examples of structural lipids include monoacyglycerols, aliphatic alcohols, and sterols. Without wishing to be bound by any theory, optimizing the formulation of LNPs by addition of such structural lipids in addition to a cationic lipid, may enhance particle stability and efficacy of nucleic acid delivery.

As used herein, the terms “monoacylglycerol” and “aliphatic alcohol” are classes of chemical compounds known to those in the art, and can include, without limitation, monoolein, 2-monoolein, citrem, oleoyl lactate, oleamide, monoelaidin, linoleic acid, monomyristolein, elaidic acid, glyceryl monoolein (GMO), monopalmitolein, monolinolein, monovaccenin, monoerucin, phytantriol (PHY), diolein, triolein, dioleoyl-glycerol, didodecyldimethylammonium bromide, dioctadecyl (dimethyl) ammonium chloride (DOAC/DODMAC) and dimethyldioctadecylammonium bromide (DODAB).

As used herein, the term “phospholipid” is any lipid that comprises a phosphate group. Phospholipids are a subset of non-cationic lipids. Without limitation, examples of phospholipids include 1,2-Dioleoyl-phosphatidic acid (DOPA), 1,2-Dioleoyl-phosphatidylglycerol (DOPG), 1,2-Distearoyl-phophatidylglycerol (DSPG), 1,2-Dioleoyl-phosphatidylethanolamine (DOPE), 1,2-distearoyl-glycero-3-phosphoethanolamine (DSPE), 1,2-Dioleoyl-phosphatidylcholine (DOPC), 1-Palmitoyl-2-oleoyl-sn-glycero-3-phosphocholine (POPC), 1,2-Dioleoyl-sn-glycero-3-phosphoserine (DOPS), 1,2-Dipalmitoylphosphatidylserine (DPPS), lyso1-hydroxy-2-oleoyl-sn-glycero-3-phosphocholine, 1,2-dioleoyl-sn-glycero-3-dihexyl-phosphocholine, and distearoylphosphatidylcholine (DSPC).

As used herein, sterols are a subgroup of steroids consisting of steroid alcohols, and are known to those in the art. Without limitation, examples of sterols include cholesterol, fecosterol, sitosterol, ergosterol, campesterol, stigmasterol, brassicasterol, and derivatives thereof such as cholestanol, tocopherol, cholestanone, cholestenone, coprostanol, cholesteryl-2′-hydroxyethyl ether, cholesteryl-4′-hydroxybutyl ether, and mixtures thereof.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the disclosure in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen. As used herein “passive immunity” refers to any immunity conferred upon a subject without administration of an antigen to the subject.

### Nucleic Acid Encapsulation

The LNP compositions described herein can be used to encapsulate nucleic acids (such as RNA). In one aspect, encapsulating agents (such as a lipid), which can be used to produce a lipid nanoparticle (LNP)-encapsulated RNA. Without intending to be bound by any theory, it is believed that the cationic or cationically ionizable lipid or lipid-like material and/or the cationic polymer combine together with the nucleic acid to form aggregates, and this aggregation results in colloidally stable particles. A lipid may be a naturally occurring lipid or a synthetic lipid. However, a lipid is usually a biological substance. Biological lipids are well known in the art, and include for example, neutral fats, phospholipids, phosphoglycerides, steroids, terpenes, lysolipids, glycosphingolipids, glucolipids, sulphatides, lipids with ether and ester-linked fatty acids and polymerizable lipids, and combinations thereof. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

In some aspects, LNPs can be designed to protect RNAs (e.g., saRNA, mRNA) from extracellular RNases and/or can be engineered for systemic delivery of the RNA to target cells. In some aspects, such LNPs may be particularly useful to deliver RNAs when RNAs are intravenously administered to a subject in need thereof. In some aspects, such LNPs may be particularly useful to deliver RNAs when RNAs are intramuscularly administered to a subject in need thereof. Accordingly, the LNPs described herein can further be administered via intravenous, intramuscular, subcutaneous, intratumoral, intranasal, or inhalation routes of administration.

A lipid nanoparticle or LNP refers to particles of any morphology generated when a cationic lipid and optionally one or more further lipids are combined, e.g. in an aqueous environment and/or in the presence of RNA. In some aspects, lipid nanoparticles are included in a formulation that can be used to deliver an active agent or therapeutic agent, such as a nucleic acid (e.g., mRNA) to a target site of interest (e.g., cell, tissue, organ, tumor, and the like). In some aspects, the lipid nanoparticles of the present disclosure comprise a nucleic acid, a polymer-lipid conjugate, a cationic lipid, and at least one structural lipid. Such lipid nanoparticles can further comprise one or more additional excipients, e.g., one or more neutral lipids, charged lipids, steroids, or combinations thereof. In some aspects, the active agent or therapeutic agent, such as a nucleic acid (e.g., mRNA), may be encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g. an adverse immune response. The nucleic acid (e.g., mRNA) or a portion thereof may also be associated and complexed with the lipid nanoparticle. A lipid nanoparticle may comprise any lipid capable of forming a particle to which the nucleic acids are attached, or in which the one or more nucleic acids are encapsulated.

In some aspects, the lipid nanoparticles may have an average particle size (as measured by their mean diameter) of about 1 to 500 nm, or about 20 to 500 nm. In some aspects, the lipid nanoparticles have a mean diameter of from about 20 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or at least, at most, exactly, or between any two of 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The term “mean diameter” refers to the mean hydrodynamic diameter of particles as measured by dynamic laser light scattering (DLS) with data analysis using the so-called cumulant algorithm, which provides as results the so-called Z-average with the dimension of a length, and the polydispersity index (PI), which is dimensionless (Koppel, D., J. Chem. Phys. 57, 1972, pp 4814-4820, ISO 13321). Here, “mean diameter,” “diameter,” or “size” for particles is used synonymously with this value of the Z-average.

LNPs described herein may exhibit a polydispersity index less than about 0.5, less than about 0.4, less than about 0.3, or about 0.2 or less. By way of example, the LNPs can exhibit a polydispersity index of at least, at most, exactly, or between any two of 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5. The polydispersity index is, in some aspects, calculated based on dynamic light scattering measurements by the so-called cumulant analysis as mentioned in the definition of the “average diameter.” Under certain prerequisites, it can be taken as a measure of the size distribution of an ensemble of nanoparticles.

In certain aspects, nucleic acids (e.g., RNAs), when present in provided LNPs, are resistant in aqueous solution to degradation with a nuclease. In some aspects, LNPs are liver-targeting lipid nanoparticles.

In certain aspects, the RNA solution and lipid preparation mixture or compositions thereof may have, have at least, have at most, have exactly, or have between any two of about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of a particular lipid, lipid type, or non-lipid component such as lipid-like materials and/or cationic polymers or an adjuvant, antigen, peptide, polypeptide, sugar, nucleic acid or other material disclosed herein or as would be known to one of skill in the art.

LNPs described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles. The term “colloid” as used herein relates to a type of homogeneous mixture in which dispersed particles do not settle out. The insoluble particles in the mixture are microscopic, with particle sizes between 1 and 1000 nm. The mixture may be termed a colloid or a colloidal suspension. Sometimes the term “colloid” only refers to the particles in the mixture and not the entire suspension.

For the preparation of colloids comprising at least one cationic or cationically ionizable lipid or lipid-like material and/or at least one cationic polymer, methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media). In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in some embodiments, formed as follows: an ethanol solution comprising lipids, such as cationic lipids and additional lipids, is injected into an aqueous solution under stirring. In some embodiments, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

In some aspects, LNP-encapsulated RNA can be produced by rapid mixing of an RNA solution described herein (e.g., the RNA product solution) and a lipid preparation described herein (comprising, e.g., at least one cationic lipid and optionally one or more other lipid components, in an organic solvent) under conditions such that a sudden change in solubility of lipid component(s) is triggered, which drives the lipids towards self-assembly in the form of LNPs. In some aspects, suitable buffering agents comprise tris, histidine, citrate, acetate, phosphate, or succinate. The pH of a liquid formulation relates to the pKa of the encapsulating agent (e.g. cationic lipid). The pH of the acidifying buffer may be at least half a pH scale less than the pKa of the encapsulating agent (e.g. cationic lipid), and the pH of the final buffer may be at least half a pH scale greater than the pKa of the encapsulating agent (e.g. cationic lipid). In some aspects, properties of a cationic lipid are chosen such that nascent formation of particles occurs by association with an oppositely charged backbone of a nucleic acid (e.g., RNA). In this way, particles are formed around the nucleic acid, which, for example, in some aspects, can result in much higher encapsulation efficiency than is achieved in the absence of interactions between nucleic acids and at least one of the lipid components.

In certain aspects, nucleic acids, when present in the lipid nanoparticles, are resistant in aqueous solution to degradation with a nuclease. Lipid nanoparticles comprising nucleic acids and their method of preparation are disclosed in, e.g., U.S. Patent Publication Nos. 2004/0142025, 2007/0042031 and PCT Pub. Nos. WO 2013/016058 and WO 2013/086373, the full disclosures of which are herein incorporated by reference in their entirety for all purposes.

Some embodiments described herein relate to compositions, methods and uses involving more than one, e.g., 2, 3, 4, 5, 6 or even more nucleic acid species such as RNA species. In an LNP formulation, it is possible that each nucleic acid species is separately formulated as an individual LNP formulation. In that case, each individual LNP formulation will comprise one nucleic acid species. The individual LNP formulations may be present as separate entities, e.g. in separate containers. Such formulations are obtainable by providing each nucleic acid species separately (typically each in the form of a nucleic acid-containing solution) together with suitable cationic or cationically ionizable lipids or lipid-like materials and cationic polymers that allow the formation of LNPs. Respective particles will contain exclusively the specific nucleic acid species that is being provided when the particles are formed (individual particulate formulations).

In some embodiments, a composition such as a pharmaceutical composition comprises more than one individual LNP formulation. Respective pharmaceutical compositions can be referred to as mixed LNP formulations. Mixed LNP formulations according to the invention can be obtained by forming, separately, individual LNP formulations, as described above, followed by a step of mixing of the individual LNP formulations. By the step of mixing, a formulation comprising a mixed population of nucleic acid-containing LNPs is obtainable. Individual LNP populations may be together in one container, comprising a mixed population of individual LNP formulations.

Alternatively, it is possible that different nucleic acid species are formulated together as a combined LNP formulation. Such formulations are obtainable by providing a combined formulation (typically combined solution) of different RNA species together with suitable cationic or cationically ionizable lipids or lipid-like materials and cationic polymers that allow the formation of LNPs. As opposed to a mixed LNP formulation, a combined LNP formulation will typically comprise LNPs that comprise more than one RNA species. In a combined LNP composition, different RNA species are typically present together in a single particle.

### Polymeric Materials

Given their high degree of chemical flexibility, polymeric materials are commonly used for nanoparticle-based delivery. Accordingly, those skilled in the art will be familiar with the types of polymers that can be used in polymer-lipid conjugates as described herein. Typically, cationic materials are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic materials useful in some embodiments herein. In addition, some investigators have synthesized polymeric materials specifically for nucleic acid delivery. Poly(P-amino esters), in particular, have gained widespread use in nucleic acid delivery owing to their ease of synthesis and biodegradability. In some embodiments, such synthetic materials may be suitable for use as cationic materials herein.

A “polymeric material,” as used herein, is given its ordinary meaning, i.e., a molecular structure comprising one or more repeat units (monomers), connected by covalent bonds. In some embodiments, such repeat units can all be identical; alternatively, in some cases, there can be more than one type of repeat unit present within the polymeric material. In some cases, a polymeric material is biologically derived, e.g., a biopolymer such as a protein. In some cases, additional moieties can also be present in the polymeric material, for example targeting moieties such as those described herein.

Those skilled in the art are aware that, when more than one type of repeat unit is present within a polymer (or polymeric moiety), then the polymer (or polymeric moiety) is said to be a “copolymer.” In some embodiments, a polymer (or polymeric moiety) utilized in accordance with the present disclosure may be a copolymer. Repeat units forming the copolymer can be arranged in any fashion. For example, in some embodiments, repeat units can be arranged in a random order; alternatively or additionally, in some embodiments, repeat units may be arranged in an alternating order, or as a “block” copolymer, i.e., comprising one or more regions each comprising a first repeat unit (e.g., a first block), and one or more regions each comprising a second repeat unit (e.g., a second block), etc. Block copolymers can have two (a diblock copolymer), three (a triblock copolymer), or more numbers of distinct blocks.

In certain embodiments, a polymeric material for use in accordance with the present disclosure is biocompatible. Biocompatible materials are those that typically do not result in significant cell death at moderate concentrations. In certain embodiments, a biocompatible material is biodegradable, i.e., is able to degrade, chemically and/or biologically, within a physiological environment, such as within the body. In certain embodiments, a polymeric material may be or comprise protamine or polyalkyleneimine, in particular protamine.

As those skilled in the art are aware term “protamine” is often used to refer to any of various strongly basic proteins of relatively low molecular weight that are rich in arginine and are found associated especially with DNA in place of somatic histones in the sperm cells of various animals (as fish). In particular, the term “protamine” is often used to refer to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin.

In some embodiments, a polyalkyleneimine comprises polyethylenimine and/or polypropylenimine. In some embodiments, the polyalkyleneimine is polyethyleneimine (PEI). In some embodiments, the polyalkyleneimine is a linear polyalkyleneimine, e.g., linear polyethyleneimine (PEI).

Cationic materials (e.g., polymeric materials, including polycationic polymers) contemplated for use herein include those which are able to electrostatically bind nucleic acid. In some embodiments, cationic polymeric materials contemplated for use herein include any cationic polymeric materials with which nucleic acid can be associated, e.g. by forming complexes with the nucleic acid or forming vesicles in which the nucleic acid is enclosed or encapsulated.

In some embodiments, particles described herein may comprise polymers other than cationic polymers, e.g., non-cationic polymeric materials and/or anionic polymeric materials. Collectively, anionic and neutral polymeric materials are referred to herein as non-cationic polymeric materials.

### Lipids & Lipid-Like Materials

According to the present disclosure, lipids and lipid-like materials may be cationic, anionic or neutral. Neutral lipids or lipid-like materials exist in an uncharged or neutral zwitterionic form at a selected pH. Generally, lipids may be divided into eight categories: fatty acids and their derivatives (including tri-, di-, monoglycerides, and phospholipids), glycerolipids, glycerophospholipids, sphingolipids, saccharolipids, polyketides, sterol lipids as well as sterol-containing metabolites such as cholesterol, and prenol lipids. Examples of fatty acids include, but are not limited to, fatty esters and fatty amides. Examples of glycerolipids include, but are not limited to, glycosylglycerols and glycerophospholipids (e.g., phosphatidylcholine, phosphatidylethanolamine, phosphatidylserine). Examples of sphingolipids include, but are not limited to, ceramides phosphosphingolipids (e.g., sphingomyelins, phosphocholine), and glycosphingolipids (e.g., cerebrosides, gangliosides). Examples of sterol lipids include, but are not limited to, cholesterol and its derivatives and tocopherol and its derivatives.

Use of structural lipids in the lipid nanoparticle may help mitigate aggregation of other lipids in the particle, and can influence the LNP morphology. Structural lipids include, but are not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. In certain embodiments, the structural lipid can be a monoacylglycerol (such as glyceryl monoolein (GMO). In other embodiments, the structural lipid can be an aliphatic alcohol (such as phytantriol (PHY)). It will be appreciated that more than one such structural lipids can be used, and the present disclosure includes any combination of such structural lipids.

The term “lipid-like material,” “lipid-like compound,” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term refers to molecules, which comprise hydrophilic and hydrophobic moieties with different structural organization, which may or may not be similar to that of lipids.

In some aspects, the RNA solution and lipid preparation mixture or compositions thereof may comprise cationic lipids, neutral lipids, cholesterol, and/or polymer-lipid conjugates (e.g., polyethylene glycol conjugated to a lipid) which form lipid nanoparticles that encompass the RNA molecules. Therefore, in some aspects, the LNP may comprise a cationic lipid and one or more excipients, e.g., one or more neutral lipids, charged lipids, steroids or steroid analogs (e.g., cholesterol), polymer-lipid conjugates (e.g. PEG-lipid), or combinations thereof. In some aspects, the LNPs encompass, or encapsulate, the nucleic acid molecules. As used herein, “encapsulate,” “encapsulated,” “encapsulation,” and grammatically comparable variants thereof mean that at least a portion of a substance is enclosed or surrounded by another material or another substance in a composition. In some aspects, a substance, such as a nucleic acid, can be fully enclosed or surrounded by another material or another substance in a composition, such as a lipid.

### Cationic Lipids

Cationic or cationically ionizable lipids or lipid-like materials refer to a lipid or lipid-like material capable of being positively charged and able to electrostatically bind nucleic acid. As used herein, a “cationic lipid” or “cationic lipid-like material” refers to a lipid or lipid like material having a net positive charge. Cationic lipids or lipid-like materials bind negatively charged nucleic acid by electrostatic interaction. Generally, cationic lipids possess a lipophilic moiety, such as a sterol, an acyl chain, a diacyl or more acyl chains, and the head group of the lipid typically carries the positive charge. Exemplary cationic lipids include one or more amine group(s) which bear the positive charge. Cationic lipids may encapsulate negatively charged RNA.

In some aspects, cationic lipids are ionizable such that they can exist in a positively charged or neutral form depending on pH. The ionization of the cationic lipid affects the surface charge of the lipid nanoparticle under different pH conditions. Without wishing to be bound by theory, this ionizable behavior is thought to enhance efficacy through helping with endosomal escape and reducing toxicity as compared with particles that remain cationic at physiological pH. For purposes of the present disclosure, such “cationically ionizable” lipids or lipid-like materials are comprised by the term “cationic lipid” or “cationic lipid-like material” unless contradicted by the circumstances.

In some embodiments, a cationic lipid may comprise from about 0.05 mol % to about 100 mol %, about 0.1 mol % to about 100 mol %, about 1 mol % to about 100 mol %, about 10 mol % to about 100 mol %, about 20 mol % to about 100 mol %, about 30 mol % to about 100 mol %, about 40 mol % to about 100 mol %, or about 50 mol % to about 100 mol % of the total lipid present in the particle. In some embodiments, a cationic lipid may be at least, at most, exactly, or between any two of 0.05 mol %, 0.1 mol %, 1 mol %, 10 mol %, 20 mol %, 30 mol %, 40 mol %, 50 mol %, 60 mol %, 70 mol %, 80 mol %, 90 mol %, or 100 mol %, or any range or value derivable therein, of the total lipid present in the particle.

The amount of the cationic lipid can also be selected taking the amount of the nucleic acid cargo into account. In one aspect, these amounts are selected such as to result in an N/P ratio of the nanoparticle(s) or of the composition in the range from at least, at most, exactly, or between any two of about 0.1, 1, 2, 3, 4, 5, 6, 7, 8, 9, 10, 11, 12, 13, 14, 15, 16, 17, 18, 19, or 20, or any range or value derivable therein. In some aspects, lipid formulations are formed at N/P ratios larger than four and up to twelve, because positively charged nanoparticles are considered favorable for transfection. In that case, RNA is considered to be completely bound to nanoparticles.

The N/P ratio is defined as the mole ratio of the nitrogen atoms (“N”) of the basic nitrogen-containing groups of the lipid to the phosphate groups (“P”) of the nucleic acid which is used as cargo. It is correlated to the charge ratio, as the nitrogen atoms (depending on the pH) are usually positively charged and the phosphate groups are negatively charged. The N/P ratio, where a charge equilibrium exists, depends on the pH. The N/P ratio may be calculated on the basis that, for example, 1 μg RNA typically contains about 3 nmol phosphate residues, provided that the RNA exhibits a statistical distribution of bases. The “N”-value of the lipid may be calculated on the basis of its molecular weight and the relative content of permanently cationic and—if present—cationizable groups.

In some aspects, the lipid nanoparticles of the present disclosure comprise one or more cationic lipids. Examples of cationic lipids include, but are not limited to: ((4-hydroxybutyl)azanediyl)bis(hexane-6,1-diyl)bis(2-hexyldecanoate); 1,2-dioleoyl-3-trimethylammonium propane (DOTAP); N,N-dimethyl-2,3-dioleyloxypropylamine (DODMA), 1,2-di-O-octadecenyl-3-trimethylammonium propane (DOTMA), 3-(N—(N′,N′-dimethylaminoethane)-carbamoyl)cholesterol (DC-Chol), dimethyldioctadecylammonium (DDAB); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-I-(cis,cis-9,12-oc-tadecadienoxy)propane (CLinDMA), 2-[5′-(cholest-5-en-3-beta-oxy)-3′-oxapentoxy)-3-dimethyl-I-(cis,cis-9′,12′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino)butanoate (DLin-MC3-DM A), N-(2-Hydroxyethyl)-N,N-dimethyl-2,3-bis(tetradecyloxy)-1-propanaminium bromide (DMRIE), (±)-N-(3-aminopropyl)-N,N-dimethyl-2,3-bis(cis-9-tetradecenyloxy)-1-propanaminium bromide (GAP-DMORIE), (±)-N-(3-aminopropyl)-N,N-dimethyl-2,3-bis(dodecyloxy)-1-propanaminium bromide (GAP-DLRIE), (±)-N-(3-aminopropyl)-N,N-dimethyl-2,3-bis(tetradecyloxy)-I-propanaminium bromide (GAP-DMRIE), N-(2-Aminoethyl)-N,N-dimethyl-2,3-bis(tetradecyloxy)-1-propanaminium bromide (bAE-DMRIE), N-(4-carboxybenzyl)-N,N-dimethyl-2,3-bis(oleoyloxy) propan-1-aminium (DOBAQ), 2-({8-[(3b)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dioleoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl)azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-I-yl)-9-((4-(dimethylaminobutanoyl)oxy) heptadecanedioate (L319), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-I-yl]ethyl]amino]dodecan-2-ol (lipidoid 02-200); (4-hydroxybutyl)azanediyl)bis(hexane-6,1-diyl)bis(2-hexyldecanoate); or heptadecan-9-yl 8-((2-hydroxyethyl) (6-oxo-6-(undecyloxy) hexyl)amino) octanoate (SM-102).

Additional cationic lipids contemplated to be within the present disclosure include those disclosed in, e.g., U.S. Pat. No. 10,166,298, and US 2020-0206362A1, the full disclosures of which are herein incorporated by reference in their entirety for all purposes.

### Additional Lipids

In certain aspects, the LNPs of the present disclosure can comprise one or more additional lipids or lipid-like materials that stabilize the formation of particles during their formation. Suitable stabilizing lipids include non-cationic lipids, e.g., neutral lipids and anionic lipids, which include the structural lipids as described herein. Without wishing to be bound by theory, optimizing the formulation of LNPs by addition of other hydrophobic moieties, such as cholesterol and lipids, in addition to an ionizable/cationic lipid or lipid-like material may enhance particle stability and efficacy of nucleic acid delivery.

As used herein, an “anionic lipid” refers to any lipid that is negatively charged at a selected pH. The term “neutral lipid” refers to any one of a number of lipid species that exist in either an uncharged or neutral zwitterionic form at physiological pH. In some embodiments, additional lipids comprise one of the following neutral lipid components: (1) a phospholipid, (2)cholesterol or a derivative thereof; or (3) a mixture of a phospholipid and cholesterol or a derivative thereof.

Representative neutral lipids include phosphatidylcholines, phosphatidylethanolamines, phosphatidylglycerols, phosphatidic acids, phosphatidylserines, ceramides, sphingomyelins, dihydro-sphingomyelins, cephalins, and cerebrosides. Exemplary phospholipids include, for example, phosphatidylcholines, e.g., diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dipalmitoylphosphatidylcholine (DPPC), dilauroylphosphatidylcholine, dioleoylphosphatidylglycerol (DOPG), dipalmitoylphosphatidylglycerol (DPPG), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), I-oleoyl-2-cholesterylhemisuccinoyl-sn-glycero-3-phosphocholine (OChemsPC), and 1-hexadecyl-sn-glycero-3-phosphocholine (C16 Lyso PC); and phosphatidylethanolamines, e.g., diacylphosphatidylethanolamines, such as dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-Icarboxylate (DOPE-mal), dipalmitoyl phosphatidyl ethanolamine (DPPE), dimyristoylphosphoethanolamine (DMPE), dilauroyl-phosphatidylethanolamine (DLPE), distearoyl-phosphatidylethanolamine (DSPE), iphytanoyl-phosphatidylethanolamine (DPyPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In one aspect, the neutral lipid is 1,2-distearoyl-sn-glycero-3phosphocholine (DSPC). In some aspects, the LNPs comprise a neutral lipid, and the neutral lipid comprises one or more of DSPC, DPPC, DMPC, DOPC, POPC, DOPE, or SM.

In various aspects, the LNPs further comprise a steroid or steroid analogue. A “steroid” is a compound comprising the following carbon skeleton:

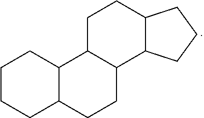 [SMILES: C1CCC2C(C1)CCC1C3CCCC3CCC21]

In certain aspects, the steroid or steroid analogue is cholesterol. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

As discussed herein, in some aspects of the present disclosure the LNPs comprise a polymer-lipid conjugate. An example of a polymer-lipid conjugate is a pegylated lipid. Pegylated lipids are known in the art and are further described herein.

In one aspect, the polyethylene glycol-lipid is PEG-2000-DMG. In one aspect, the polyethylene glycol-lipid is PEG-c-DOMG). In another aspect, the polyethylene glycol-lipid is 2-[(polyethylene glycol)-2000]-N,N-ditetradecylacetamide (PEG-DMA). In other aspects, the LNPs comprise a PEGylated diacylglycerol (PEG-DAG) such as 1-(monomethoxy-polyethyleneglycol)-2,3-dimyristoylglycerol (PEG-DMG), a PEGylated phosphatidylethanoloamine (PEG-PE), a PEG succinate diacylglycerol (PEG-S-DAG) such as 4-O-(2′,3′-di(tetradecanoyloxy)propyl-1-O-((o-methoxy(polyethoxy)ethyl)butanedioate (PEG-S-DMG), a PEGylated ceramide (PEG-cer), or a PEG dialkoxypropylcarbamate such as co-methoxy(polyethoxy)ethyl-N-(2,3di(tetradecanoxy)propyl)carbamate or 2,3-di(tetradecanoxy)propyl-N-(u>-methoxy(polyethoxy)ethyl)carbamate. PEG-lipids are disclosed in, e.g., U.S. Pat. No. 9,737,619, the full disclosure of which is herein incorporated by reference in its entirety for all purposes.

In various aspects, the molar ratio of the cationic lipid to the pegylated lipid ranges from about 100:1 to about 20:1, e.g., from about 20:1, 25:1, 30:1, 35:1, 40:1, 45:1, 50:1, 55:1, 60:1, 65:1, 70:1, 75:1, 80:1, 85:1, 90:1, 95:1, or 100:1, or any range or value derivable therein.

In certain aspects, the PEG lipid is present in the LNP in an amount from about 0.1 to about 10 mole percent (mol %) (e.g., at least, at most, exactly, or between any two of 0.1, 0.5, 1, 2, 3, 4, 5, 6, 7, 8, 9, or 10 mol %), relative to the total lipid content of the nanoparticle. In one aspect, the PEG lipid is present in the LNP in an amount from about 1 to about 5 mole percent. In one aspect, the PEG lipid is present in the LNP in about 1 mole percent or about 1.5 mole percent.

Without wishing to be bound by any particular theory, the relative amount of lipids in the LNPs of the present disclosure may affect important nucleic acid particle characteristics, such as charge, particle size, stability, tissue selectivity, and bioactivity of the nucleic acid. Accordingly, the relative amounts of lipids within the LNPs of the present disclosure are described herein.

Prolonged Immune Response from Vaccinations

The slow cellular internalization of GMO:Chol LNPs poses benefits for sustained antigen production during vaccination with mRNA-LNPs, which is emerging to be useful for improve immunogenicity. A slow delivery has recently been shown useful for difficult vaccination targets for example, HIV (J. H. Lee et al. Nature 609, pp. 998-1004 (2022). Current vaccination strategies enable immunogenic responses to develop against specific viruses that last for hours to days. Humans have naturally evolved to develop longer lasting immune responses against natural infections, some of which can last for weeks. This is of particular importance in cancer vaccination where the immune system requires a prolonged priming response to boost naturally occurring tumor antigens (Grunwitz & Kranz, mRNA Cancer Vaccines—Messages that Prevail; Current Topics in Microbiology and Immunology, SpringerLink Vol. 405 (2017)). Improving vaccinations to induce longer lasting immune responses is of critical importance. Sustained delivery of antigens or sustained production of antigens via slow internalization of mRNA could improve adaptive immune responses of vaccinations (Ou et al., Adv. Drug Del. Rev. 187, 11401 (2022)).

### LNPs for Controlled Release

LNPs that have a sustained, or controlled, release profile can be used for a variety of therapeutic and prophylactic purposes, such as for cancer and/or immunotherapy. For example, for applications involving the sustained release of mRNA, therapeutic areas that may benefit would involve a local administration (e.g. intradermal, intramuscular, intratumoral etc.) See, e.g., Ziller et al. Mol. Pharmaceutics 15, 2, pp. 642-651 (2018). Some therapeutic areas in which controlled/sustained release of mRNA could be applied include but are not limited to: local cancers that require intratumoral injection e.g. pancreatic cancer (localized not unresectable) (Narayanan et al., J. Immunother Cancer 2023 11: e006133. doi: 10.1136/jito-2022-006133), and skin cancers etc. Sustained releasing mRNA-LNP combined with delivery of a cytokine via intratumoral injection could be beneficial towards cancer immunotherapy (Liu et al., J. Controlled Release Vol 345 pp. 306-313 (2022)).

Accordingly, the LNPs of the present disclosure may allow sustained or delayed release of the polynucleotide, primary construct, or mRNA (eg, after intramuscular or subcutaneous injection). The altered release profile for the polynucleotide, the primary construct, or the mRNA may result, for example, in the translation of an encoded protein over an extended period of time. The LNPs of the present disclosure can also be used to increase the stability of the polynucleotide, primary construct, or mRNA. Biodegradable polymers have previously been used to protect nucleic acids other than mRNA from degradation and have been shown to produce sustained release of payloads in vivo (Rozema et al., Proc Natl Acad Sci USA. 2007 104:12982-12887; Sullivan et al., Expert Opin Drug Deliv. 20107:1433-1446. The pharmaceutical compositions of the present disclosure may be sustained release formulations. Sustained release formulations may be for subcutaneous administration.

Accordingly, the present disclosure provides LNPs that have a delayed release profile of the RNA contained therein. For example, in one aspect, an LNP provides a release profile that is 0.001% or less, 0.005% or less, 0.01% or less, 0.05% or less, 0.1% or less, 0.5% or less, 1% or less, 5% or less, 10% or less, 20% or less, 30% or less, 40% or less, 50% or less, 60% or less, 70% or less, 80% or less, 90% or less, or 95% or less, of the release profile of a comparator LNP that does not have a delayed release profile. For example, such comparator LNPs can be any of those disclosed in WO 2013/086373 and WO 2021/213924, or the standard LNP control, DSPC:Chol (10:37.5 [denoted as 198]) LNP as described in Example 9 herein.

### EXAMPLES

The present disclosure is described in greater detail by way of specific examples. The following examples are offered for illustrative purposes and are not intended to limit the disclosure in any manner.

### Example 1: Glyceryl Monoolein LNP Formulation—One Helper Lipid

LNPs were formed via microfluidic mixing of an aqueous and lipidic phase, using a Nanoassemblr device. For the lipidic phase, a cationic lipid (Dlin-MC3-DMA), helper lipid (GMO) and PEG-lipid (DMG- or DOPE-PEG) were combined in the ratio of lipid components as shown in Table 1. For example, Dlin-MC3-DMA ranged from 1.5-50%, GMO ranged from 48.5-97% and PEG lipid was maintained at 1.5%. For the aqueous phase, modified mRNA was diluted in citrate buffer, pH 5.5. The aqueous to lipid ratio was 3:1, at a total flow rate of 12 mL/min. After the LNPs were formed, they were dialyzed using 10 mM Tris buffer, pH 7.5 overnight. Post dialysis, 300 mM sucrose was added to stabilize the LNPs and then they were stored at −80 °C.

Dynamic light scattering was used to determine the particle size which ranged from 114-161 nm with a polydispersity index ranging from 0.10 to 0.15. The encapsulation efficiency was determined using a RNA assay kit and ranged from 42-94%. RNA integrity was determined by fragment analyzer, using capillary gel electrophoresis and the value for one of the formulations was 80%. The amount of RNA degradation determined from the area of the curve of late migrating species (LMS) and the value of one of the formulations was 6%.

### Example 2: Glyceryl Monoolein and Cholesterol LNP Formulation—Two Helper Lipids

LNPs were formed via microfluidic mixing of an aqueous and lipidic phase, using a Nanoassemblr device. For the lipidic phase, a cationic lipid (Dlin-MC3-DMA), two helper lipids (GMO and cholesterol) and PEG-lipid (DMG- or DOPE-PEG) were combined in the ratio of lipid components as shown in Table 1. For example, Dlin-MC3-DMA ranged from 1.5-50 mol %, GMO ranged from 10-77 mol %, cholesterol ranged from 10-77 mol % and PEG lipid was maintained at 1.5 mol %. For the aqueous phase, modified mRNA was diluted in citrate buffer, pH 5.5. The aqueous to lipid ratio was 3:1, at a total flow rate of 12 mL/min. After the LNPs were formed, they were dialyzed using 10 mM Tris buffer, pH 7.5 overnight. Post dialysis, 300 mM sucrose was added to stabilize the LNPs and then they were stored at −80 °C.

Dynamic light scattering was used to determine the particle size which ranged from 74-175 nm with a polydispersity index ranging from 0.09 to 0.21. When the cholesterol content was less than 71.4 mol %, LNPs formed within the desired size range of <120 nm. The encapsulation efficiency was determined by a RNA assay kit and ranged from 76-99%. RNA integrity was determined by fragment analyzer, using capillary gel electrophoresis and the values ranged from 80-84%. The amount of RNA degradation determined from the area of the curve of late migrating species (LMS) and values ranged from 2-7%.

TABLE 1
Glyceryl monoolein (GMO) lipid nanoparticles (LNPs) investigation, detailing the type of lipid used,
the associated composition ratio in mol % and nitrogen to phosphate (N/P) ratio. The corresponding
critical quality attributes (CQAs) of lipid nanoparticles are displayed. Data reported as the average
of two individual experiments.

| | | | | | Criteria | | | | |
|---|---|---|---|---|---|---|---|---|---|
| | | | | | 30-120 nm | <0.3 | >80% | >60% | <15% |
| Glyceryl monoolein (GMO) LNP investigation | | | | | Quantification | | | | |
| Variable | | | | | Particle | | | RNA | |
| Cationic | Helper | PEG- | | | size | | % EE | integrity | LMS |
| Lipid | Lipid | Lipid | Ratio | N/P | (nm) | PDI | (RNA) | (% POI) | (%) |
| Dlin- | GMO | DMG- | 50:48.5:1.5 | 6 | 161 | 0.12 | 42 | NA | NA |
| MC3- | | PEG | | | | | | | |
| DMA | | | | | | | | | |
| Dlin- | GMO | DOPE- | 28.5:70:1.5 | 3.42 | 122 | 0.13 | 94 | 80 | 6 |
| MC3- | | PEG | | | | | | | |
| DMA | | | | | | | | | |
| Dlin- | GMO | DMG- | 8.5:90:1.5 | 1.02 | 121 | 0.15 | 86 | NA | NA |
| MC3- | | PEG | | | | | | | |
| DMA | | | | | | | | | |
| Dlin- | GMO | DMG- | 1.5:97:1.5 | 0.18 | 114 | 0.10 | 61 | NA | NA |
| MC3- | | PEG | | | | | | | |
| DMA | | | | | | | | | |
| Dlin- | Chol:GMO | DOPE- | 50:10:38.5:1.5 | 6 | 173 | 0.16 | 86 | NA | NA |
| MC3- | | PEG | | | | | | | |
| DMA | | | | | | | | | |
| Dlin- | Chol:GMO | DOPE- | 28.5:14.4:55.6:1.5 | 3.42 | 114 | 0.17 | 92 | 81 | 6 |
| MC3- | | PEG | | | | | | | |
| DMA | | | | | | | | | |
| Dlin- | Chol:GMO | DOPE- | 8.5:18.6:71.4:1.5 | 1.02 | 70 | 0.11 | 96 | 82 | 4 |
| MC3- | | PEG | | | | | | | |
| DMA | | | | | | | | | |
| Dlin- | Chol:GMO | DOPE- | 1.5:20:77:1.5 | 0.18 | 100 | 0.10 | 92 | 84 | 2 |
| MC3- | | PEG | | | | | | | |
| DMA | | | | | | | | | |
| Dlin- | GMO:Chol | DMG- | 50:10:38.5:1.5 | 6 | 141 | 0.14 | 92 | 80 | 7 |
| MC3- | | PEG | | | | | | | |
| DMA | | | | | | | | | |
| Dlin- | GMO:Chol | DOPE- | 28.5:14.4:55.6:1.5 | 3.42 | 74 | 0.09 | 99 | 81 | 5 |
| MC3- | | PEG | | | | | | | |
| DMA | | | | | | | | | |
| Dlin- | GMO:Chol | DOPE- | 8.5:18.6:71.4:1.5 | 1.02 | 175 | 0.21 | 76 | NA | NA |
| MC3- | | PEG | | | | | | | |
| DMA | | | | | | | | | |
| Dlin- | DSPC:GMO | DMG- | 50:10:38.5:1.5 | 6 | 149 | 0.14 | 63 | NA | NA |
| MC3- | | PEG | | | | | | | |
| DMA | | | | | | | | | |
| Dlin- | DSPC:GMO | DMG- | 8.5:18.6:71.4:1.6 | 1.02 | 83 | 0.18 | 86 | 82 | 4 |
| MC3- | | PEG | | | | | | | |
| DMA | | | | | | | | | |
| Dlin- | DSPC:GMO | DMG- | 1.5:20:77:1.5 | 0.18 | 87 | 0.18 | 90 | 81 | 5 |
| MC3- | | PEG | | | | | | | |
| DMA | | | | | | | | | |
| Dlin- | DSPC:Chol | DMG- | 50:10:38.5:1.5 | 6 | 117 | 0.18 | 95 | 76 | 10 |
| MC3- | | PEG | | | | | | | |
| DMA | | | | | | | | | |
| Dlin- | DSPC:Chol | DOPE- | 28.5:14.4:55.6:1.5 | 3.42 | 102 | 0.16 | 98 | 65 | 21 |
| MC3- | | PEG | | | | | | | |
| DMA | | | | | | | | | |

NA = not available.

### Example 3: Phytantriol LNP Formulation—One Helper Lipid

LNPs were formed via microfluidic mixing of an aqueous and lipidic phase, using a Nanoassemblr device. A different helper lipid, phytantriol (PHY) was used in the formulations. For the lipidic phase, a cationic lipid (Dlin-MC3-DMA), helper lipid (PHY) and PEG-lipid (DMG- or DOPE-PEG) were combined in the ratio of lipid components as shown in Table 2. For example, Dlin-MC3-DMA ranged from 1.5-50 mol %, PHY ranged from 48.5-97 mol % and PEG lipid was maintained at 1.5 mol %. For the aqueous phase, modified mRNA was diluted in citrate buffer, pH 5.5. The aqueous to lipid ratio was 3:1, at a total flow rate of 12 mL/min. After the LNPs were formed, they were dialyzed using 10 mM Tris buffer, pH 7.5 overnight. Post dialysis, 300 mM sucrose was added to stabilize the LNPs and then they were stored at −80 °C.

Dynamic light scattering was used to determine the particle size which ranged from 97 nm to greater than 1000 nm with a polydispersity index ranging from 0.03 to 1.0. When the amount of PHY was less than 90 mol %, the particle size of the LNPs was less than 300 nm. The encapsulation efficiency was determined by a RNA assay kit and ranged from 77-98%. RNA integrity was determined by fragment analyzer, using capillary gel electrophoresis and ranged from 52-81%. The amount of RNA degradation determined from the area of the curve of late migrating species (LMS) and the value for PHY at 70 mol % was 5%, while it increased to 26% when 48.5 mol % PHY was used in the formulations.

### Example 4: Phytantriol and Glyceryl Monoolein LNP Formulation—Two Helper Lipids

LNPs were formed via microfluidic mixing of an aqueous and lipidic phase, using a Nanoassemblr device. Two different helper lipids (glyceryl monoolein and phytantriol) were used in the formulations. For the lipidic phase, a cationic lipid (Dlin-MC3-DMA), two helper lipids (GMO and PHY) and PEG-lipid (DMG- or DOPE-PEG) were combined in the ratio of lipid components as shown in Table 2. For example, Dlin-MC3-DMA ranged from 1.5-50 mol %, GMO ranged from 14.4-77 mol %, PHY ranged from 10-77 mol % and PEG lipid was maintained at 1.5 mol %. For the aqueous phase, modified mRNA was diluted in citrate buffer, pH 5.5. The aqueous to lipid ratio was 3:1, at a total flow rate of 12 mL/min. After the LNPs were formed, they were dialyzed using 10 mM Tris buffer, pH 7.5 overnight. Post dialysis, 300 mM sucrose was added to stabilize the LNPs and then they were stored at −80 °C.

Dynamic light scattering was used to determine the particle size which ranged from 81-140 nm with a polydispersity index ranging from 0.04 to 0.27. The encapsulation efficiency was determined by a RNA assay kit and ranged from 22-99%. RNA integrity was determined by fragment analyzer, using capillary gel electrophoresis and the values ranged from 53-78%. The amount of RNA degradation determined from the area of the curve of late migrating species (LMS) and values ranged from 4-29%.

TABLE 2
Phytantriol (PHY) and GMO LNPs investigation, detailing the type of lipid used,
the associated composition ratio in mol % and nitrogen to phosphate (N/P) ratio.
The corresponding critical quality attributes (CQAs) of lipid nanoparticles (LNPs)
are displayed. Data reported as the average of two individual experiments.

| | | | | | Criteria | | | | |
|---|---|---|---|---|---|---|---|---|---|
| | | | | | 30-120 nm | <0.3 | >80% | >60% | <15% |
| Phytantriol (PHY) and GMO LNP investigation | | | | | Quantification | | | | |
| Variable | | | | | Particle | | | RNA | |
| Cationic | Helper | PEG- | | | size | | % EE | integrity | LMS |
| Lipid | Lipid | Lipid | Ratio | N/P | (nm) | PDI | (RNA) | (% POI) | (%) |
| Dlin-MC3- | PHY | DMG- | 50:48.5:1.5 | 6 | 97 | 0.12 | 98 | 52 | 26 |
| DMA | | PEG | | | | | | | |
| Dlin-MC3- | PHY | DMG- | 28.5:70:1.5 | 3.42 | 90 | 0.03 | 97 | 81 | 5 |
| DMA | | PEG | | | | | | | |
| Dlin-MC3- | PHY | DMG- | 8.5:90:1.5 | 1.02 | 257 | 0.08 | 77 | NT | NA |
| DMA | | PEG | | | | | | | |
| Dlin-MC3- | PHY | DMG- | 1.5:97:1.5 | 0.18 | >1000 | 1.00 | NA | NA | NA |
| DMA | | PEG | | | | | | | |
| Dlin-MC3- | PHY:Chol | DMG- | 50:10:38.5:1.5 | 6 | 133 | 0.17 | 97 | 40 | 36 |
| DMA | | PEG | | | | | | | |
| Dlin-MC3- | PHY:Chol | DOPE- | 28.5:14.4:55.6:1.5 | 3.42 | >1000 | 1.00 | NA | NA | NA |
| DMA | | PEG | | | | | | | |
| Dlin-MC3- | Chol:PHY | DOPE- | 28.5:14.4:55.6:1.5 | 3.42 | 62 | 0.11 | 99 | NA | NA |
| DMA | | PEG | | | | | | | |
| Dlin-MC3- | Chol:PHY | DOPE- | 8.5:18.6:71.4:1.5 | 1.02 | 112 | 0.06 | 98 | NA | NA |
| DMA | | PEG | | | | | | | |
| Dlin-MC3- | Chol:PHY | DOPE- | 1.5:20:77:1.5 | 0.18 | 142 | 0.10 | 56 | 72 | 10 |
| DMA | | PEG | | | | | | | |
| Dlin-MC3- | DSPC:PHY | DOPE- | 1.5:20:77:1.5 | 0.18 | 294 | 0.18 | 15 | 77 | 4 |
| DMA | | PEG | | | | | | | |
| Dlin-MC3- | PHY:GMO | DMG- | 50:10:38.5:1.5 | 6 | 140 | 0.27 | 59 | 53 | 29 |
| DMA | | PEG | | | | | | | |
| Dlin-MC3- | PHY:GMO | DMG- | 28.5:14.4:55.6:1.5 | 3.42 | 98 | 0.11 | 83 | 78 | 8 |
| DMA | | PEG | | | | | | | |
| Dlin-MC3- | PHY:GMO | DMG- | 8.5:18.6:71.4:1.5 | 1.02 | 123 | 0.17 | 91 | 55 | 27 |
| DMA | | PEG | | | | | | | |
| Dlin-MC3- | PHY:GMO | DMG- | 1.5:20:77:1.5 | 0.18 | 90 | 0.09 | 22 | NA | NA |
| DMA | | PEG | | | | | | | |
| Dlin-MC3- | GMO:PHY | DOPE- | 28.5:14.4:55.6:1.5 | 3.42 | 94 | 0.16 | 99 | 78 | 6 |
| DMA | | PEG | | | | | | | |
| Dlin-MC3- | GMO:PHY | DOPE- | 8.5:18.6:71.4:1.5 | 1.02 | 89 | 0.04 | 95 | NA | NA |
| DMA | | PEG | | | | | | | |
| Dlin-MC3- | GMO:PHY | DMG- | 1.5:20:77:1.5 | 0.18 | 81 | 0.09 | 80 | 67 | 4 |
| DMA | | PEG | | | | | | | |

NA = not available.

### Example 5: Stability of LNPs at Refrigerated and Ambient Temperatures

Exemplary LNPs were chosen based on meeting at least three out of four critical quality attributes, where at least the particle size and RNA integrity meet the desired critical quality attribute criteria. The desired criteria are defined as; particle size <300 nm, PDI<0.3, % encapsulation efficiency >80%, % RNA integrity >60% and % LMS<15%.

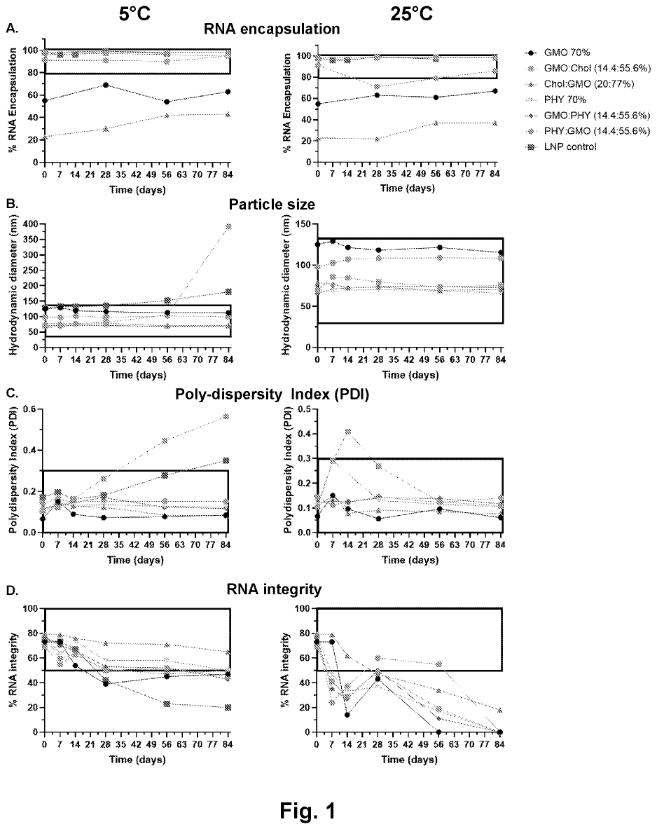

The six exemplary LNP formulations (GMO 70 mol %, GMO:Chol 14.4:55.6 mol %, Chol:GMO 20:77 mol %, PHY 70 mol %, GMO:PHY 14.4:55.6 mol % and 10 PHY:GMO 14.4:55.6 mol %) were all prepared at a larger scale (6 g) and aliquoted into 500 μL tubes. Samples were split across controlled temperature conditions (5 °C and 25 °C) at ambient humidity, mimicking refrigerator and room temperature, respectively. At designated timepoints of 0, 7, 14 days as well as 1, 2 and 3 months, an individual sample was removed and tested for particle size, polydispersity, RNA encapsulation and RNA integrity. From both investigations, the six exemplary LNPs were taken into stability studies to assess the CQAs over a period of time at 5 °C and 25 °C (see FIG. 1).

### Example 6: Morphology of LNPs

Cryo-electron microscopy (EM) was used to visualize the morphologies of the lipid nanoparticles. First, a 3 μL sample of LNPs was applied to a Lacey carbon, 300 mesh, Au grid then blotted in a controlled environment (5 °C, 100%) for 60 seconds and then plunged into liquid nitrogen cooled liquid ethane to vitrify. The morphologies of the LNPs were different amongst formulations. The standard DSPC:cholesterol (10:38.5 mol %) LNPs had irregular spherical-like structures with occurrences of “blebs” from the particle surfaces (Error! Reference source not found. A). This structural morphology has previously been reported on by Yanez Arteta et al. (PNAS 2018, 115:E3351). Standard DSPC:cholesterol LNPs also had distinctive shell layer on the particles, indicative of a lamellar-type bilayer. In comparison, 70 mol % GMO LNPs population were spherical and densely packed within the core as observed by the darker colored particles in Error! Reference source not found. B. GMO:cholesterol (14.4:55.6 mol %) LNPs were less dense in color, but had particle populations with well-defined straight edges, similar to hexagonal shapes, as demonstrated by the white box outlines in Error! Reference source not found. C. The core of these particles was similar in density to the shell, showing uniformity throughout the particle. Increasing the GMO content and decreasing the cholesterol content (i.e. cholesterol:GMO 22:77 mol % LNPs) resulted in “bleb” formation, with the inability to distinguish between single particle morphologies, as observed in Error! Reference source not found. D. PHY 70 mol % LNPs were again different to GMO 70 mol % LNPs, where particles had both cubic-like shapes (red boxes) as well as uniquely defined graph lines within the core (black boxes, Error! Reference source not found. E). For PHY:GMO 14.4:55.6 mol % LNPs, the density increased with the occurrence of the cubic shaped particles (Error! Reference source not found. F). The dense cubic shaped particles observed for PHY:GMO 14.4:55.6 mol % is further indicative of improved thermostability at 5 °C and 25 °C.

### Example 7: Morphology of LNPs-Small Angle X-Ray Scattering

Small angle x-ray scattering (SAXS) experiments were conducted on Brookhaven National Laboratories National Synchrotron Light Source II. LNPs were measured in glass capillaries with a sample to detector distance of 3.687 m, giving a q range 0.005 to 3.1 Å⁻¹. The wavelength of the X-rays λ was 0.8206 Å. The intensity versus q graphs were plotted to determine the index spacing of major peaks in order to correlate global structural configurations of the LNPs.

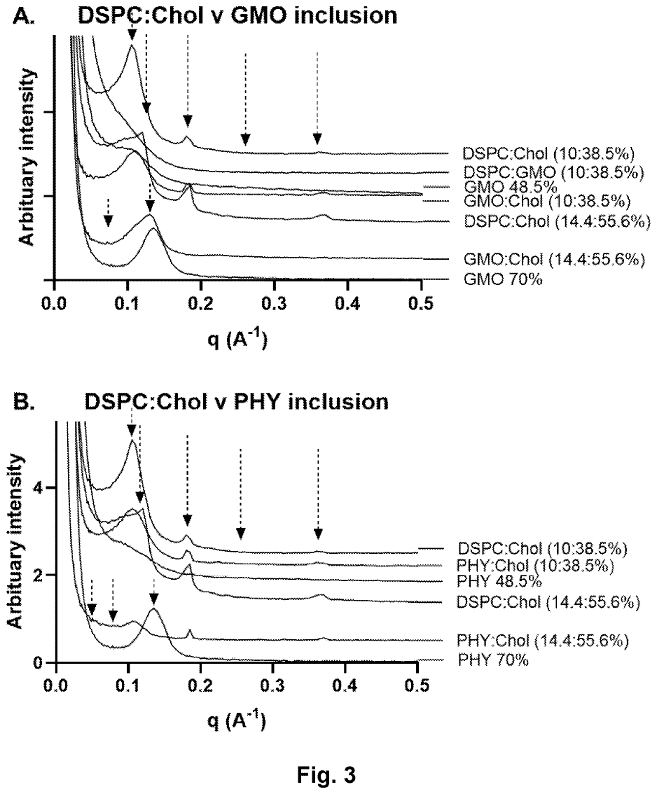

SAXS diffractograms in Error! Reference source not found. highlight peaks indexed 1, 2 and 3 spacings for DSPC:Cholesterol 10:37.5% LNPs, indicative of the lamellar phase. Increasing the amount of DSPC:Cholesterol to 14.4:55.6% in the LNPs, while decreasing the cationic lipid, MC3, resulted in a shift of the Bragg peaks to 1, √3 and 2 spacings in FIG. 3A, highlighting a phase shift to the inverse hexagonal phase. When GMO replaced DSPC, forming GMO:Chol 10:37.5% LNPs, the inverse hexagonal phase was also demonstrated by Bragg peaks at the 1:√3:2 spacing ratio (FIG. 3A).

A similar shift to Bragg peaks at spacing ratio 1:√3:2, was observed in FIG. 3 for when PHY replaced DSPC, forming PHY:Chol 10:37.5% LNPs. When the ratio of GMO:Chol was increased to 14.4:55.6% and the MC3 was decreased, another shift occurred compared to the standard DSPC:Chol, where a broad Bragg peak was observed at: √3 (FIG. 3B). This broad peak could not be resolved into single peaks but is expected to contain multiple peaks, indicative of the inverse bi-continuous cubic phase, where the lipid bilayers display triply periodic minimal surface geometry and interweaved between two connecting water channels (Meikle T. G. et al. Colloids and Surfaces B: Biointerfaces, 152, pp. 143-151 (2017)). This structure represents a tightly packed arrangement of lipids. A similar diffractogram indicative of the bi-continuous cubic phase was also observed for three-component system using GMO as the structural lipid, at 47.5% and 70% mole ratio. For PHY based systems, increasing the amount of PHY:Chol to 14.4:55.6% mol maintained an inverse hexagonal phase structure. However, a three-component PHY system at 47.5% mol and 70% mol had diffractograms that shifted to a broad peak at: \sqrt{3}, suggesting a transition to the inverse bi-continuous cubic phase.

### Example 8: Morphology of LNPs—NMR and Cryo-EM

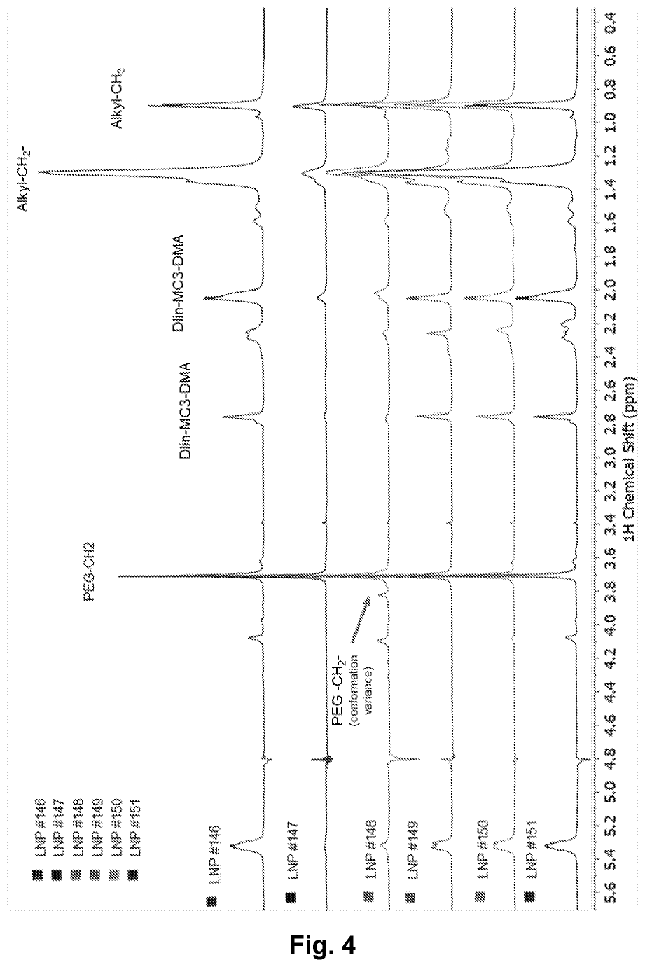

The one-dimensional proton nuclear magnetic resonance (1D 1H NMR) spectra of different LNPs is displayed in FIG. 4, and provides information on the chemical identity of the surface lipids of the LNPs.

Samples were measured as intact LNPs. Samples were diluted with 0.2M×DPBS, and spiked 10% D₂O, 0.05% trisodium phosphate. Different excitation sculpting pulse sequences were used to render the 1D 1H NMR spectra including; (1) 1d1H, sequence: zgesgp −H 90 degree pulse and (2) 1d1H, sequence: PGSTE (stebpesgp1s1d)−T2 filter to suppress excipients signal.

Surface PEG methylene signals are well resolved on the surface of all LNPs. LNPs with PHY 70% mol (#149) and GMO:PHY 14.4:55.6% mol (#150) show very similar surface lipid profiles, likely attributable to the fact that similar amount of PHY are within the formulation. Similarly, LNPs with GMO 70% (#146) and PHY:GMO 14.4:55.6% (#151) also show very similar surface lipid profiles, for the same reason as PHY, and differ in peak positions only by the chemical shifts of GMO compared to PHY.

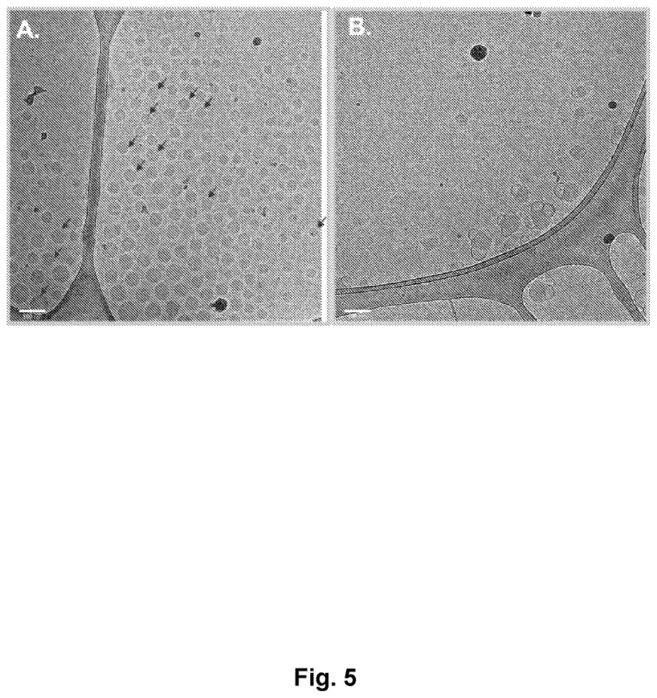

LNPs with GMO:Chol 14.4:55.6% mol (#147) distinctly shows surface lipid profiles with significantly lowest peak intensity (FIG. 3). It suggests the less mobile surface lipids and likely a more rigid particle. Electron microscopy (EM) shows particles that have stripe patterns that may be caused by the combination of GMO and cholesterol. In FIG. 4A, the EM also demonstrated that many particles in this sample are slightly cubic in shape (arrow), which corresponds with the SAXS diffractograms. GMO and cholesterol play a role as structural lipids such that the higher amount of cholesterol (55.6% mol) combined with GMO promotes the particle shape to change. For LNPs with GMO:Chol 22:77% mol (#148), there is a smaller PEG-methylene peak at 3.8 ppm that is likely associated with surface PEG changes (FIG. 4). This peak correlates to the high abundance of blebs observed in EM images in FIG. 5B. Both high levels of glyceryl monooleate and cholesterol likely contribute to bleb formation.

### Example 9: LNP Cell Uptake and Cytoplasmic Distribution

Hela cells were seeded (1×10⁴ cells/well) in 96 well plates and incubated with mRNA-LNPs. At a variety of time points (1 h, 4 h and 24 h), different quantification techniques were used to determine LNP (1) uptake/association, (2) internalization and (3) mRNA translation. For mRNA-LNP uptake/association, quantitative reverse transcription polymerase chain reaction (RT-qPCR) was employed to quantify the amount of mRNA within the cell after 1 h and 4 h of incubation. For mRNA internalization into the cytoplasm (i.e. release from the endosome), single-molecule Fluorescence in situ Hybridization (smFISH) was used at 4 h and 24 h post incubation. Lastly, for mRNA translation, a standard immunofluorescence assay was used to determine the total cell count, the amount of HA positive cells and the mean fluorescence intensity, 24 h post incubation with mRNA-LNPs, where the mRNA encoded for the HA-influenza virus protein.

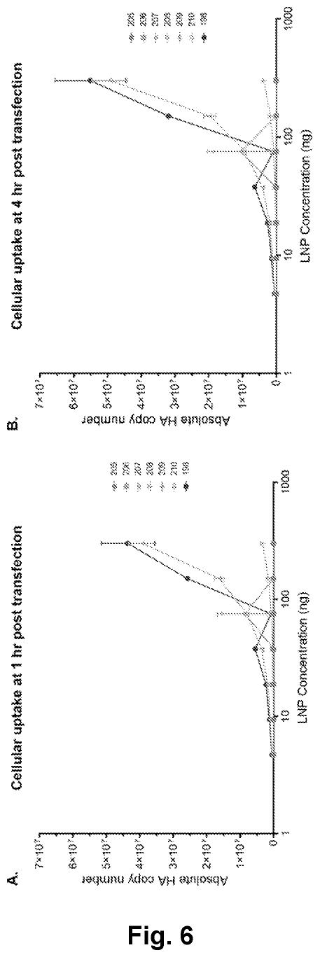

For LNP cell uptake, GMO:Chol (14.4:55.6 [denoted as 207]) LNPs rapidly associated with the cell, where up to approximately 4×10⁷ HA copies were quantified within the cell 1 h post transfection, as demonstrated in FIG. 6. This was equivalent to the standard LNP control, DSPC:Chol (10:37.5 [denoted as 198]) LNP. For both of these LNPs the cellular uptake/association was retained at the same level at 4 h post transfection. There was a minimum of 0.5×10⁷ HA copies quantified for Chol:GMO (22:77%)/208 LNP at 1 h and 4 h, indicating potential cell association, although this was close to the limit of detection. For other LNPs, including GMO (70%)/#205, PHY (70%)/#206, GMO:PHY (14.4:55.6)/#209 and PHY:GMO (14.4:55.6)/#210, minimal to no cell association of mRNA was detected.

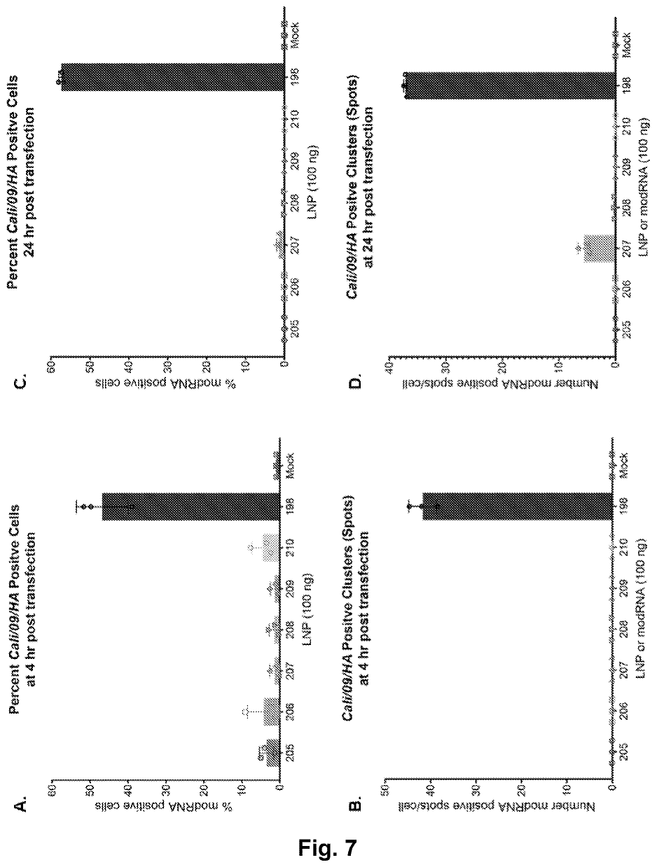

In the next step of the transfection process, cellular internalization of the mRNA-LNPs by imaging cytoplastic distribution was examined at 4 h and 24 h post transfection. At 4 h, out of the six LNPs tested, only the standard LNP, DSPC:Chol (10:37.5%)/198 resulted in cytoplasmic distribution of the mRNA, quantified as 50% modRNA positive cells (FIG. 7A), with approximately 40 mRNA positive spots per cell (FIG. 7B). Following 24 h, the amount of mRNA distributed within the cytoplasm remained consistent per cell for the standard LNP. However, the cytoplasmic distribution of mRNA increased from GMO:Chol (14.4:55.6%)/#207 LNP, with less than 5% modRNA positive cells (FIG. 7C), and approximately 5 mRNA positive spots per cell (FIG. 7D). This indicated a slower internalization of mRNA from GMO:Chol (14.4:55.6)/#207 LNPs compared to the standard LNP control. Further time points were not taken to continue this observation. All other LNPs did not demonstrate any cytoplasmic distribution at 4 h or 24 h.

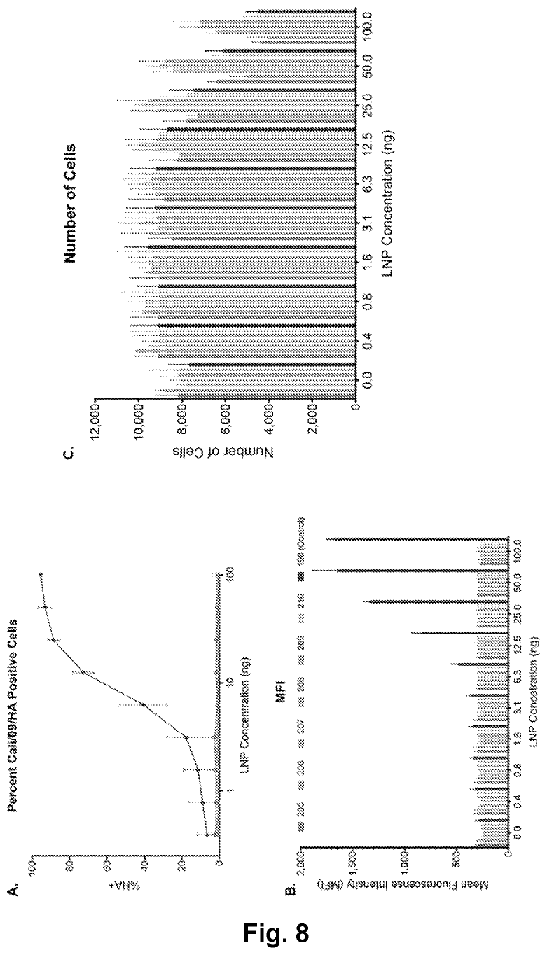

Consequently, a dose response transfection was only observed with DSPC:Chol (10:37.5%)/#198 LNP between 0.39 and 100 ng mRNA-LNP, with an EC50 of approximately 9 ng, as observed from the percentage of positive HA antigen and mean fluorescence intensity graphs in FIGS. 8A and B, respectively. All other LNPs did not demonstrate any response at 24 h post transfection. The limited response is likely related to the limited cytoplasmic distribution of the novel LNPs at 4 h post transfection. While GMO:Chol (14.4:55.6%)/#207 LNP began to distribute within the cytoplasm at 24 h, the level of mRNA is unlikely sufficient to drive translation to HA protein. While the response was not measured beyond 24 h, it is suggested that longer transfection periods may enable a response from the GMO:Chol (14.4:55.6%) LNP.

Cell viability was sequentially assessed during the transfection assay by determine the number of cells remaining post 24 h incubation. LNPs containing GMO:Chol 14.4:55.6 (207), Chol:GMO 22:77 (208), and GMO:PHY 14.4:55.6 (209) were less cytotoxic at higher concentrations (>12.5 ng) compared to the standard LNP DSPC:Chol 10:37.5 (198) (FIG. 8C).

1-24. (canceled)

25. A lipid nanoparticle (LNP) comprising:
(a) at least one nucleic acid;
(b) at least one polyethylene glycol (PEG)-lipid conjugate in an amount from about 1.0 to about 2.0 mol % of the total lipid in the LNP;
(c) at least one cationic lipid in an amount from about 1 to about 30 mol % of the total lipid in the LNP; and
(d) at least one structural lipid selected from glyceryl monoolein (GMO) and phytantriol (PHY), wherein each of said at least one structural lipids is present in an amount from about 1 to about 90 mol % of the total lipid in the LNP.

26. The LNP of claim 25, wherein the PEG-lipid conjugate is selected from: dimyristoylphosphatidylcholine-polyethylene glycol-2000 (DMPC-PEG); 1,2-dimyristoyl-rac-glycero-3-methoxypolyethylene glycol-2000 (DMG-PEG); 1,2-dioleoyl-sn-glycero-3-phosphoethanolamine-N-[amino(polyethylene glycol)-2000] ammonium salt (DOPE-PEG); 1,2-distearoyl-sn-glycero-3-phosphoethanolamine-poly(ethylene glycol)-2000 (DSPE-PEG); mPEG-N,N-ditetradecylacetamide (ALC-0159); and oleoyl polyethylene glycol 2000.

27. The LNP of claim 25, wherein the at least one cationic lipid is selected from: (6Z,9Z,28Z,31Z)-heptatriaconta-6,9,28,31-tetraen-19-yl 4-(dimethylamino)butanoate (Dlin-MC3-DMA); [(4-hydroxybutyl)azanediyl]di(hexane-6,1-diyl)bis(2-hexyldecanoate) (ALC-0315); 2-[2,2-bis[(9Z,12Z)-octadeca-9,12-dienyl]-1,3-dioxolan-4-yl]-N,N-dimethylethanamine (DLin-KC2-DMA); 1,2-dilinoleyloxy-n,n-dimethyl-3-aminopropane (DLinDMA); N,N-dimethyl-2,3-bis[(Z)-octadec-9-enoxy]propan-1-amine (DODMA); 5-carboxyspermylglycinedioctadecylamide (DOGS); and 1,2-dioleoyl-3-dimethylammonium-propane (DODAP).

28. The LNP of claim 27, wherein the at least one cationic lipid is Dlin-MC3-DMA.

29. The LNP of claim 25, further comprising at least one additional structural lipid that is not GMO or PHY, wherein said at least one additional structural lipid is present in an amount from about 10 to about 75 mol % of the total lipid present in the LNP.

30. The LNP of claim 29, wherein: the at least one cationic lipid is present in an amount of about 28.5 mol % of the total lipid present in the LNP; GMO is present in an amount of about 14.4 mol % of the total lipid present in the LNP; the at least one additional structural lipid that is not a monoacylglycerol or an aliphatic alcohol is cholesterol and is present in an amount of about 55.6 mol % of the total lipid present in the LNP; and the at least one polymer-lipid conjugate is DOPE-PEG and is present in an amount of about 1.5 mol % of the total lipid present in the LNP.

31. The LNP of claim 29, wherein: the at least one cationic lipid is present in an amount of about 1.5 mol % of the total lipid present in the LNP; GMO is present in an amount of about 77 mol % of the total lipid present in the LNP; the at least one additional structural lipid that is not a monoacylglycerol or an aliphatic alcohol is cholesterol and is present in an amount of about 20 mol % of the total lipid present in the LNP; and the at least one polymer-lipid conjugate is DOPE-PEG and is present in an amount of about 1.5 mol % of the total lipid present in the LNP.

32. The LNP of claim 25, wherein: the at least one cationic lipid is present in an amount of about 28.5 mol % of the total lipid present in the LNP; PHY is present in an amount of about 70 mol % of the total lipid present in the LNP; and the at least one polymer-lipid conjugate is DMG-PEG and is present in an amount of about 1.5 mol % of the total lipid present in the LNP.

33. The LNP of claim 25, wherein: the at least one cationic lipid is present in an amount of about 28.5 mol % of the total lipid present in the LNP; GMO is present in an amount of about 14.4 mol % of the total lipid present in the LNP; PHY is present in an amount of about 55.6 mol % of the total lipid present in the LNP; and the at least one polymer-lipid conjugate is DOPE-PEG and is present in an amount of about 1.5 mol % of the total lipid present in the LNP.

34. The LNP of claim 25, wherein: the at least one cationic lipid is present in an amount of about 28.5 mol % of the total lipid present in the LNP; PHY is present in an amount of about 14.4 mol % of the total lipid present in the LNP; GMO is present in an amount of about 55.6 mol % of the total lipid present in the LNP; and the at least one polymer-lipid conjugate is DMG-PEG and is present in an amount of about 1.5 mol % of the total lipid present in the LNP.

35. The LNP of claim 25, wherein: the at least one cationic lipid is present in an amount of about 28.5 mol % of the total lipid present in the LNP; GMO is present in an amount of about 70 mol % of the total lipid present in the LNP; and the at least one polymer-lipid conjugate is DOPE-PEG and is present in an amount of about 1.5 mol % of the total lipid present in the LNP.

36. The LNP of claim 25, wherein the at least one nucleic acid is mRNA.

37. A pharmaceutical composition comprising the LNP of claim 25 and a pharmaceutically acceptable carrier.

38. The pharmaceutical composition of claim 37, which is formulated for intravenous administration.

39. A method for delivering a nucleic acid to a cell comprising contacting the cell with the LNP of claim 25.
